SEQUENCE LISTING (2 sequences)

SEQ ID NO: 1 - protein, 53 aa, Homo sapiens
  MISC_FEATURE: Epidermal Growth Factor
NSDSECPLSHDGYCLHDGVCMYIEALDKYACNCVVGYIGERCQYRDLKWWELR

SEQ ID NO: 2 - protein, 208 aa, homo sapiens
  MISC_FEATURE: Proheparin-binding EGF-like growth factor precursor
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

(19) United States
(12) Patent Application Publication
(10) Pub. No.: US 2017/0072016 A1
(43) Pub. Date: Mar. 16, 2017

(54) TREATMENT OF NEONATAL BRAIN INJURY WITH HB-EGF

(71) Applicant: CHILDREN'S NATIONAL MEDICAL CENTER, Washington, DC (US)

(72) Inventors: Vittorio GALLO, Washington, DC (US); Joseph SCAFIDI, Ellicott City, MD (US)

(73) Assignee: CHILDREN'S NATIONAL MEDICAL CENTER, Washington, DC (US)

(21) Appl. No.: 15/123,991

(22) PCT Filed: Mar. 6, 2015
(86) PCT No.: PCT/US15/19169

§ 371 Date: Sep. 6, 2016

Related U.S. Application Data
(60) Provisional application No. 61/949,065, filed on Mar. 6, 2014.

(51) Int. Cl. A61K 38/18; A61M 11/00; A61M 1/00; A61K 9/00

(52) U.S. Cl. CPC A61K 38/1808; A61K 9/0043; A61M 11/007; A61M 1/0003; A61M 2210/0618

(57) ABSTRACT

Methods for treating brain injury caused by or associated with hypoxia or a hypoxic state, especially neonatal brain injury, using heparin-binding EGF-like growth factor.

### CROSS-REFERENCE TO RELATED APPLICATIONS

This application claims benefit of U.S. Provisional Application No. 61/949,065, filed Mar. 6, 2014 which is hereby incorporated by reference in its entirety.

### STATEMENT REGARDING FEDERALLY SPONSORED RESEARCH OR DEVELOPMENT

This invention was made with government support under National Institute of Health grants K08NS073793; NSADA K12NS052159; K08NS069815; P01NS062686; R01NS045702; R01NS056427; P30HD040677; R01MH067528; P30 NS05219; and R01MH067528. The government may have certain rights in the invention.

### BACKGROUND OF THE INVENTION

Field of the Invention

Treatment of brain injury with epidermal growth factor (“EGF”).

Description of Related Art

There are no clinically relevant treatments available that improve function in the growing population of very preterm infants (less than 32 weeks' gestation) with neonatal brain injury. Diffuse white matter injury (DWMI) is a common finding in these children and results in chronic neurodevelopmental impairments^{1,2}. As shown recently, failure in oligodendrocyte progenitor cell maturation contributes to DWMI³. We demonstrated previously that the epidermal growth factor receptor (EGFR) has an important role in oligodendrocyte development⁴. The amino acid sequence of heparin-binding EGF-like growth factor is known and is given by Accession No. NP_001936, version NP_001936.1 or by GI:450341 (SEQ ID NO: 1).

### BRIEF SUMMARY OF THE INVENTION

A method for treating a subject having a brain injury caused by or associated with hypoxia comprising administering heparin-binding EGF-like growth factor, a functional fragment thereof, or a variant thereof to the subject. A subject in need of treatment includes neonates such as term and preterm infants both male and female. A preterm infant may be born or delivered at a gestational age of 37 weeks or less, for example, at 37, 36, 35, 34, 33, 32, 31, 30, 29, 28, 27, 26, 25, 24, 23 or fewer weeks gestation. The ranges described herein, such as that above, include all intermediate subranges and values. Preferred subjects include preterm infants delivered at less than 32 weeks gestation. Other preferred subjects include neonates or preterm infants a neonate who have been subjected to a hypoxic state, who are hypoxic, or who are at risk of hypoxia. However, this method may be used to treat other subjects having insufficient lung function, for example, neonates with incomplete or abnormal lung development or who lack a sufficient amount of lung surfactant. A subject may exhibit diffuse white matter injury or neurodevelopmental impairment.

For the method disclosed above, the heparin-binding EGF-like growth factor, a functional fragment thereof, or a variant thereof may be isolated from a natural source, from cell culture, or produced recombinantly. A variant or functional fragment of a heparin-binding EGF-like growth factor that has an amino acid sequence that is at least 80%, 85%, 90%, 95%, 97.5%, 99% or 100% identical to Accession No. NP_001936, version NP_001936.1 (SEQ ID NO: 2) may be administered. The ranges described herein, such as that above, include all intermediate subranges and values.

The method disclosed herein may be performed by administering the heparin-binding EGF-like growth factor, a functional fragment thereof, or a variant to a subject within 0, 0.25, 0.5, 1, 2, 3, 4, 5, 6, 7, 8, 9, 10, 12, 15, 16, 20, 24, 30, 36, 40, 48, 50, 60 or 72 hrs of birth or brain insult or injury. The ranges described herein, such as that above, include all intermediate subranges and values.

The heparin-binding EGF-like growth factor, functional fragment, or variant is administered by a route and in an amount effective to prevent, ameliorate, or treat brain insult or injury. It may be introduced topically, onto a mucus membrane, into the nose (intranasally), into an airway, bronchi or lungs (intrapulmonarily), systemically, for example, parenterally, intravenously, intramuscularly, subcutaneously, or into the central or peripheral nervous system. Preferably, it is administered in a non-invasive or in the least invasive way, for example, by intranasal administration. It may be administered with an appropriate pharmaceutically carrier or excipient and/or in combination with other active ingredients, such as an anti-inflammatory drug, a natural or synthetic pulmonary surfactant, or other drugs used to treat neonates, especially preterm infants. It may be administered as a prodrug, such as a peptide prodrug, modified or conjugated to moieties that facilitate its passage across physiological barriers such as the endothelial blood-brain barrier or the epithelial blood-cerebrospinal fluid barrier. A dosage of ranging from 1 to 2,000 μg/kg body weight, from 10 to 500 μg/kg body weight, or 50 to 200 μg/kg body weight may be administered.

The invention is also directed to a method for diminishing ultrastructural abnormalities caused by or associated with hypoxia in subject comprising administering heparin-binding EGF-like growth factor, a functional fragment thereof, or a variant thereof to said subject. A subject in need of treatment includes neonates such as term and preterm infants both male and female. A preterm infant may be born or delivered at a gestational age of 37 weeks or less, for example, at 37, 36, 35, 34, 33, 32, 31, 30, 29, 28, 27, 26, 25, 24, 23 or fewer weeks gestation. Preferred subjects include preterm infants delivered at less than 32 weeks gestation. This method may be used to treat other subjects having insufficient lung function, for example, neonates with incomplete or abnormal lung development or who lack a sufficient amount of a lung surfactant. A subject may exhibit diffuse white matter injury or neurodevelopmental impairment.

Another aspect of the invention is a method for decreasing oligodendroglia death, enhancing generation or regeneration of new oligodendrocytes from progenitor cells, and/or promoting cellular recovery in white matter after hypoxia, in a subject comprising administering heparin-binding EGF-like growth factor, a functional fragment thereof, or a variant thereof to said subject. A subject in need of treatment includes neonates such as term and preterm infants both male and female. A preterm infant may be born or delivered at a gestational age of 37 weeks or less, for example, at 37, 36, 35, 34, 33, 32, 31, 30, 29, 28, 27, 26, 25, 24, 23 or fewer weeks gestation. Preferred subjects include preterm infants delivered at less than 32 weeks gestation. Other preferred subjects include neonates or preterm infants a neonate who have been subjected to a hypoxic state, who are hypoxic, or who are at risk of hypoxia. This method may be used to treat other subjects having insufficient lung function, for example, neonates with incomplete or abnormal lung development or who lack a sufficient amount of lung surfactant. A subject may exhibit diffuse white matter injury or neurodevelopmental impairment.

Another aspect of the invention is a method for alleviating behavioral deficits associated with hypoxic brain injury on white-matter-specific paradigms or promoting functional recovery in a subject comprising administering heparin-binding EGF-like growth factor, a functional fragment thereof, or a variant thereof to a subject. A subject in need of treatment includes neonates such as term and preterm infants both male and female. A preterm infant may be born or delivered at a gestational age of 37 weeks or less, for example, at 37, 36, 35, 34, 33, 32, 31, 30, 29, 28, 27, 26, 25, 24, 23 or fewer weeks gestation. Preferred subjects include preterm infants delivered at less than 32 weeks gestation. Other preferred subjects include neonates or preterm infants a neonate who have been subjected to a hypoxic state, who are hypoxic, or who are at risk of hypoxia and who exhibit behavioral deficits associated with hypoxic brain injury on a white-matter-specific paradigm.

Another aspect of the invention is a composition comprising heparin-binding EGF-like growth factor, a functional fragment thereof, or a variant thereof in combination with a solution, carrier or excipient that facilitates its uptake by the peripheral nervous system or by the central nervous system (“CNS”). Such a composition may comprise a heparin-binding EGF-like growth factor, a functional fragment thereof, or a variant thereof in combination with a solution, carrier or excipient that facilitates its uptake after intranasal administration by the CNS. Such compositions include solutions, suspensions, emulsions in oily or aqueous carriers such as O/W or W/O emulsions, gels, pastes, and sustained-release formulations and may comprise one or more additional ingredients such as suspending, stabilizing, buffers, preservatives, or dispersing agents. The active ingredients may be encapsulated or provided in particulate form, such as in microcapsules or within nanoparticles.

The invention is also directed to devices that store, meter, and deliver or dispense an effective dose of heparin-binding EGF-like growth factor, a functional fragment thereof, or a variant thereof to a subject in need thereof. Examples of such products include an intranasal or aspirator device containing heparin-binding EGF-like growth factor, a functional fragment thereof, or a variant thereof and, optionally, a syringe or pump, spray nozzle, atomizer, a channel or conduit, and/or storage container which may be pressurized or unpressurized.

Specific aspects or embodiments of the invention include, without limitation the following:

1. A method for treating a subject having a brain injury caused by or associated with hypoxia comprising administering heparin-binding EGF-like growth factor, a functional fragment thereof, or a variant thereof to said subject.

2. The method of embodiment 1, wherein the subject is a neonate.

3. The method of embodiment 1, wherein the subject is a preterm infant.

4. The method of embodiment 1 wherein the subject is a preterm infant less than 32-weeks gestation.

5. The method of embodiment 1, wherein the subject has diffuse white matter injury (“DWMI”).

6. The method of embodiment 1, wherein the subject exhibits neurodevelopmental impairment.

7. The method of embodiment 1, wherein the heparin-binding EGF-like growth factor, a functional fragment thereof, or a variant thereof was produced recombinantly.

8. The method of embodiment 1, comprising administering a variant or functional fragment of heparin-binding EGF-like growth factor that has an amino acid sequence that is at least 90% identical to Accession No. NP_001936, version NP_001936.1 (SEQ ID NO: 2).

9. The method of embodiment 1, comprising administering a variant or functional fragment of heparin-binding EGF-like growth factor that has an amino acid sequence that is at least 95% identical to that described by Accession No. NP_001936, version NP_001936.1 (SEQ ID NO: 2).

10. The method of embodiment 1, comprising administering an EGF-like growth factor comprising the amino acid sequence of SEQ ID NO: 2.

11. The method of embodiment 1, wherein heparin-binding EGF-like growth factor, a functional fragment thereof, or a variant thereof is administered within 0, 0.25, 0.5, 1, 2, 3, 4, 5, 6, 7, 8, 9, 10, 12, 15, 16, 20, 24, 30, 36, 40, 48, 50, 60 or 72 hrs of brain injury.

12. The method of embodiment 1, comprising administering 10 to 1,000 mg/kg of heparin-binding EGF-like growth factor, a functional fragment thereof, or a variant thereof to the subject.

13. The method of embodiment 1, comprising administering the heparin-binding EGF-like growth factor, a functional fragment thereof, or a variant thereof into the central nervous system.

14. The method of embodiment 1 comprising administering the heparin-binding EGF-like growth factor, a functional fragment thereof, or a variant thereof intranasally.

15. A method for diminishing ultrastructural abnormalities caused by or associated with hypoxia in subject comprising administering heparin-binding EGF-like growth factor, a functional fragment thereof, or a variant thereof to said subject.

16. The method of embodiment 15, wherein the subject is a neonate.

17. The method of embodiment 15, wherein the subject is a preterm infant.

18. A method for decreasing oligodendroglia death and/or enhancing generation of new oligodendrocytes from progenitor cells in a subject comprising administering heparin-binding EGF-like growth factor, a functional fragment thereof, or a variant thereof to said subject.

19. The method of embodiment 18, wherein said subject is a neonate who has been subjected to a hypoxic state, who is hypoxic, or who is at risk of hypoxia.

20. The method of embodiment 18, wherein said subject is a preterm infant who has been subjected to a hypoxic state, who is hypoxic, or who is at risk of hypoxia.

21. A method for alleviating behavioral deficits associated with hypoxic brain injury on a white-matter-specific paradigm or promoting functional recovery in a subject comprising administering heparin-binding EGF-like growth factor, a functional fragment thereof, or a variant thereof to said subject.

22. The method of embodiment 21, wherein said subject is a neonate who has been subjected to a hypoxic state, who is hypoxic, or who is at risk of hypoxia.

23. The method of embodiment 21, wherein said subject is a preterm infant who has been subjected to a hypoxic state, who is hypoxic, or who is at risk of hypoxia.

24. A composition comprising heparin-binding EGF-like growth factor, a functional fragment thereof, or a variant thereof in combination with a solution, carrier or excipient that facilitates its uptake by the CNS after intranasal administration.

25. An intranasal spray or aspirator device comprising the composition of embodiment 24 and, optionally, pump, atomizer, a channel or conduit, and/or storage container which may be pressurized or unpressurized. A metered dosing sprayer or aspirator. Delivery devices for a composition according to the invention may also include metered dose inhalers (MDIs), dry powder inhalers (DPIs), and nebulizers.

### BRIEF DESCRIPTION OF THE SEVERAL VIEWS OF THE DRAWING

FIG. 1: Enhanced EGFR expression in oligodendrocyte lineage cells prevents oligodendrocyte and myelin loss, and ultrastructural and behavioural deficits caused by neonatal hypoxia. a-d, Confocal images of white matter immunostained for MBP. Hyp, hypoxia-treated group; Nx, normoxia group. Scale bar, 50 μm. e, Western blot of white matter tissue (n=4 mice per group and per age except P18 normoxia and hypoxia-exposed mice, n=5; one-way analysis of variance (ANOVA), Bonferroni post-hoc test for individual comparisons). f-h, Number of Rep⁺Olig2⁺ and Rep⁺CCl⁺ cells. i, Number of newly generated oligodendrocytes in white matter. f-i, n=4 mice per group and per age; one-way ANOVA, Bonferroni post-hoc test for individual comparisons. j-m, Electron microscopy images from P60 white matter. Scale bar, 0.5 μm. n, o, Scatter plots of g ratios of individual axons relative to axon diameters (n=3 mice per group; one-way ANOVA of all four groups with post-hoc unpaired t-tests). p, Vₘₐₓ (m min⁻¹) over time (days) on the complex wheel (linear regression comparison of slopes between all four groups; post-hoc comparison of individual days between groups: Nx Rep, n=12; Nx Rep-hEGFR, n=9; Hyp Rep, n=12; Hyp Rep-hEGFR, n=10). q, Naive mice were tested on the 2-cm and 1-cm-width inclined beam-walking task (Poisson multiple regression analysis; Nx Rep, n=8; Nx Rep-hEGFR, n=10; Hyp Rep, n=10; Hyp Rep-hEGFR, n=10). r, A separate group of naive mice was assessed (Poisson multiple regression analysis; Nx Rep, n=9; Nx Rep-hEGFR, n=8; Hyp Rep, n=10; Hyp Rep-hEGFR, n=9). Line graph and histograms are presented as means±standard error of the mean (s.e.m.). *P<0.05; **P<0.01.

FIG. 2: EGFR activity is crucial for white matter recovery after neonatal hypoxia. a, Protocol of gefitinib and BrdU administration. Different groups are indicated. Hyp, hypoxia treated; Nx, normoxia. b, Western blot of white matter shows that gefitinib decreased pEGFR in the normoxia group and prevented the increase in pEGFR after hypoxia (n=5 mice per group; one-way ANOVA, Bonferroni post-hoc test for individual comparisons). c, Counts of Rep⁺Olig2⁺ and Rep⁺CCl⁺ cells in white matter. d, Gefinitib increased cell apoptosis in Nx and Hyp groups. e, f, Gefitinib decreased Rep⁺NG2⁺ OPCs (e) and oligodendrocyte-lineage cell proliferation (f) in normoxia mice and prevented hypoxia-induced increase in OPC and oligodendrocyte-lineage cell proliferation. g, Long-term effects of gefinitib on Rep⁺Olig2⁺ and Rep⁺CCl⁺ cells. h, Gefinitib decreased newly generated Rep⁺CCl⁺ oligodendrocytes in normoxia mice and prevented oligodendrogenesis after hypoxia. i, Gefitinib prevented recovery of CNPase and MBP expression after hypoxia. c-i, n=4 mice per all groups and per age; one-way ANOVA, Bonferroni post-hoc test for individual comparisons. All histograms are presented as means±s.e.m. *P<0.05; **P<0.01.

FIG. 3: Intranasal HB-EGF accelerates oligodendrocyte regeneration and promotes cellular recovery in white matter after neonatal hypoxia. a, Confocal image of the corpus callosum (CC) in a preterm infant, highlighting an Olig2⁺EGFR⁺ cell (box). Scale bars, 50 μm. b, Protocol of intranasal HB-EGF/BrdU administration and tissue collection. Different groups are indicated. Hyp, hypoxia treated; Nx, normoxia. c, Number of white matter Rep⁺Olig2⁺ and Rep⁺CCl⁺ cells in HB-EGF-treated mice. d, HB-EGF attenuated or prevented the effects of hypoxia on oligodendrocyte apoptosis. e, HB-EGF had an additive effect on hypoxia-induced increase of Rep⁺NG2⁺ OPCs at P15, but not at P18. f, HB-EGF had an additive effect on hypoxia-induced increase of OPC proliferation. g, HB-EGF promoted oligodendrogenesis after hypoxia at P18. h, Fate mapping of OPCs (PDGFαR-CreER; Z/EG (GFP) mouse) demonstrated that oligodendrogenesis occurred from PDGFαR⁺ cells. c-h, n=4 mice per group and per age; one-way ANOVA, Bonferroni post-hoc test for individual comparisons. i, Removal of EGFR in PDGFαR-expressing OPCs (PDGFαR-CreER; EGFR^{fl/fl}; Z/EG mouse) caused a decrease in NG2⁺ OPCs after hypoxia and prevented the effects of HB-EGF (n=4 mice per group except hypoxia HB-EGF PDGFαR-CreER; EGFR^{fl/fl} group, n=3;

one-way ANOVA of all four groups with post-hoc unpaired t-tests). j, HB-EGF promoted recovery in white matter MBP and PLP protein levels after hypoxia (western blot; n=6 mice per group except Nx HB-EGF, n=4; one-way ANOVA, Bonferroni post-hoc test for individual comparisons). All histograms are presented as means±s.e.m. *P<0.05; **P<0.01.

FIG. 4: Intranasal HB-EGF promotes ultrastructural, physiological and behavioural recovery in white matter after neonatal hypoxia. a-f, Electron microscopy images of P30 white matter (a-d) (Scale bar, 0.5 μn) and scatter plots of g ratios of individual axons relative to axon diameters (e, f) (n=3 mice per group; one-way ANOVA of all four groups with post-hoc unpaired t-tests). Hyp, hypoxia treated; Nx, normoxia. g-j, Ex vivo DTI analysis (representative averaged FA maps) shows that HB-EGF attenuates hypoxia-induced reduction of FA values in the corpus callosum, cingulum and external capsule (arrows point to regions of interest; n=5 for each group). k, CAP extracellular recordings. Representative waveforms and histograms show that the Hyp HB-EGF group had larger M amplitudes compared with the Hyp saline group (n=7 per group per age except P60 Nx HB-EGF n=5; one-way ANOVA, Bonferroni post-hoc test for individual comparisons). 1, Ex vivo measurements of white matter NAA using ¹H-nuclear magnetic resonance (¹H-NMR) spectroscopy. Representative ¹H-NMR spectra showing the NAA peak. At P18 there was significantly less NAA in the Hyp saline group compared with the Hyp HB-EGF group (n=5 per group per age; one-way ANOVA, Bonferroni post-hoc test for individual comparisons, except P11 unpaired t-test). m, On the complex wheel, Hyp HB-EGF mice performed similarly to the Nx saline group (linear regression comparison of slopes between all three groups and post-hoc comparison of individual days between groups; Nx saline, n=8; Hyp saline, n=12; Hyp HB-EGF, n=10). n, o, On the inclined beam-walking task, HB-EGF treatment (P11-P14) decreased the number of foot slips observed in hypoxia-exposed mice (Poisson multiple regression analysis; all groups were n=8, except for the P30 Hyp HB-EGF group, n=9). p, Delayed HB-EGF administration at P18-P21 resulted in no difference in foot slips (Poisson multiple regression analysis; Nx saline and Nx HB-EGF n=7; Hyp saline and Hyp HB-EGF, n=8). All histograms are presented as means±s.e.m. *P<0.05; **P<0.01.

FIG. 5 (Extended Data FIG. 1): Hypoxia results in a significant increase in EGF levels in the white matter. The white matter was dissected out at P11, P15 and P18 in normoxia (Nx)- and hypoxia (Hyp)-exposed CNP-EGFP (Rep) and CNP-EGFP-hEGFR (Rep-hEGFR) mice. At P11, in both Hyp groups, there was a significant increase in EGF levels, as measured by ELISA. There was no significant difference between the two Hyp groups, indicating that overexpression of EGFR in oligodendrocyte lineage cells does not modify endogenous EGF levels. At P15 and P18, there was no difference between all four groups. All histograms are presented as mean absorption (optical density (OD)) relative to total protein concentration±s.e.m. *P<0.05; **P<0.01 (P11 and P15, n=4 mice per group and per age; P18, n=3 mice per group; one-way ANOVA, Bonferroni post-hoc test for individual comparisons).

FIG. 6 (Extended Data FIG. 2): Enhanced EGFR expression in oligodendrocyte lineage cells prevents oligodendrocyte death and promotes proliferation of OPCs in white matter. a-d, Representative ×40 confocal images of Rep⁺PI⁺ cells from normoxia-exposed and hypoxia=exposed white matter at P11 in Rep and Rep-hEGFR mice. e, At P11 and P18, hypoxia resulted in a significant increase in the number of oligodendrocyte cells undergoing apoptosis (Rep⁺Casp3⁺). Enhanced EGFR expression prevented this increase at all time points, except P60 where no difference was evident. Comparison of the Hyp Rep with the Hyp Rep-hEGFR groups demonstrates that hEGFR in oligodendrocyte lineage cells is protective against apoptosis induced by hypoxia (n=4 mice per group and per age; one-way ANOVA, Bonferroni post-hoc test for individual comparisons). f, PI was injected intraperitoneally 1 h before mice were killed. A significant increase in the number of Rep⁺PI⁺ oligodendrocyte cells indicated membrane disruption contributing to cell death (n=3 mice per group; one-way ANOVA, Bonferroni post-hoc test for individual comparisons). g-j, Representative ×40 confocal images of Rep⁺NG2⁺ OPCs from normoxia-exposed and hypoxia-exposed white matter at P18 in Rep and Rep-hEGFR mice. k, At P11 and P18, Rep-hEGFR Nx mice had more NG2 OPCs compared with the Rep Nx group. Hypoxia resulted in a significant increase in the number of Rep⁺NG2⁺ OPCs in both Rep and Rep-hEGFR groups; however, overexpression of hEGFR did not have an additive effect (n=4 mice per group and per age; one-way ANOVA, Bonferroni post-hoc test for individual comparisons). 1, At P11 and P18, the Rep-hEGFR group had more Rep⁺Ki67⁺ cells; however, this did not reach significance (P>0.05). Hypoxia resulted in enhanced oligodendrocyte-lineage proliferation at P11 and P18, but hEGFR overexpression did not have a significantly additive effect when compared with the Rep Hyp group. k, 1, n=4 mice per group and per age; one-way ANOVA, Bonferroni post-hoc test for individual comparisons. m, More OPCs were in a proliferative state in Rep-hEGFR Nx mice compared with Rep Nx. Hypoxia enhanced OPC proliferation, and overexpression of hEGFR resulted in a significantly additive increase compared to the Rep Hyp group (n=3 mice per group; one-way ANOVA, Bonferroni post-hoc test for individual comparisons). All histograms are presented as means±s.e.m. Scale bars, 50 μm (a-d, g-j). ̂P=0.05; *P<0.05; **P<0.01.

FIG. 7 (Extended Data FIG. 3): The inclined beam-walking task requires normal subcortical white matter. a-c This study was performed to test the hypothesis that the inclined beam-walking task is a good assessment of subcortical white matter function. To test this hypothesis, we tested a well-established model of subcortical white matter demyelination induced by bilateral lysolecithin injections in 8-week-old C57BL/6J male and female mice. Animals were tested at 5 days after surgical intervention—which is a time period when demyelination is at its maximum—to determine whether subcortical white matter integrity is important in this behavioural task. Control mice received bilateral injections of normal saline using the same coordinates as the lysolecithin group. a, b, Bilateral demyelination was confirmed after testing by removal of brains and immunohistochemical analysis of corpus callosum. Only mice that had clear bilateral lesions on microscopic examination were included in the behavioural analysis (n=3 mice were excluded). c, The lysolecithin-injected mice had a marginally significant or very significant increase in average number of foot slips on the 2-cm- and-1-cm-wide inclined beam, respectively (two-tailed Mann-Whitney test, n=6 per group). d, We wanted to determine whether the Rep (CNP-EGFP) transgenic mice performance on the inclined beam-walking task was similar to C57BL/6 mice (wild type). No difference in performance was evident between the two different lines of male mice. We found that the Rep and wild-type mice performed similarly in either normoxic or hypoxic conditions (Poisson multiple regression analysis; Nx Rep, n=7; Nx wild type, n=11; Hyp Rep, n=8; Hyp wild type, n=11). All histograms are presented as means±s.e.m. ̂P=0.05; *P<0.05; **P<0.01.

FIG. 8 (Extended Data FIG. 4): Inhibition of EGFR prevents expansion of progenitor cells in the developing white matter and after hypoxia. a-d, Representative ×40 confocal images of subcortical white matter Sox2⁺ cells, a transcription factor expressed in proliferating multipotential neural progenitor cells. e, Gefitinib, a specific EGFR inhibitor, resulted in a significant decrease in the number of white matter Sox2-expressing cells compared with normoxia-exposed vehicle-treated mice. After hypoxia, there was a significant expansion of Sox2⁺ cells in the white matter compared with the Nx vehicle group; however, this expansion was prevented by gefitinib (Hyp gefitinib) (Hyp vehicle versus Hyp gefitinib, P<0.01) (n=4 for each group; one-way ANOVA, Bonferroni post-hoc test for individual comparisons). f-i, Representative ×40 confocal images of subcortical white matter Ascl1⁺ cells (Mash1) in the Ascl1-eGFP transgenic mice. Ascl1 is a proneural transcription factor expressed in proliferating multipotential neural progenitor cells. j, Similar to above, gefitinib resulted in a significant decrease in the total number of Ascl1-eGFP⁺ cells in the white matter compared with normoxia-exposed vehicle-treated mice. Hypoxia resulted in a significant expansion in the number of Ascl1-eGFP⁺ cells, which gefitinib prevented (Hyp vehicle versus Hyp gefitinib, P<0.05) (n=4 for each group; one-way ANOVA, Bonferroni post-hoc test for individual comparisons). All histograms are presented as means±s.e.m. *P<0.05; **P<0.01. a-d, f-i, Scale bars, 50 μm.

FIG. 9 (Extended Data FIG. 5): Intranasal HB-EGF does enter the brain and activates EGFRs in oligodendrocyte lineage cells. a-c, Saline or HB-EGF was administered intranasally once in P11 mice, which were then killed at 1, 5, 15 and 30 min after administration. a, Western blot analysis was performed on microdissected white matter probing for actin, HB-EGF and pEGFR (Tyr 1068 phosphorylation site). In the saline group, no HB-EGF was detected in the white matter and no change in pEGFR was detected. In the mice that received HB-EGF, the HB-EGF protein was detected at 5 min and increased up to 30 min. The pEGFR signal steadily increased at 5, 15 and 30 min after HB-EGF administration. b, c, The line graphs represent relative abundance of protein compared with actin (n=3 for each time point and condition). Line graphs are presented as means±s.e.m. d, A normoxia-treated P11 mouse was administered HB-EGF and killed 30 min later. Immunohistochemistry of pEGFR was performed. In the white matter, there were several Rep⁺pEGFR⁺DAPI⁺ cells, indicating that oligodendrocyte lineage cells express activated EGFR (pEGFR). Shown is a ×40 representative image of the white matter demonstrating Rep⁺pEGFR⁺DAPI⁺ cells. e, In this set of experiments, Hyp Rep mice received either intranasal saline or HB-EGF from P11-P14 (FIG. 3b). The subcortical white matter was microdissected at P15 and CNP-eGFP cells were FACS purified. Western blot analysis was performed to probe for pEGFR. The western blot demonstrates that a more robust signal for pEGFR was present in the Hyp HB-EGF group, relative to actin (n=4 for each group of 2-3 pooled brains).

FIG. 10 (Extended Data FIG. 6): Intranasal HB-EGF treatment increases the number of oligodendrocyte lineage cells derived from PDGFαR-expressing OPCs. a, PDGFαR-CreER; Z/EG transgenic mice were divided into four groups. Saline or HB-EGF were administered intranasally. Intraperitoneal injections of tamoxifen were administered at P12, P13 and P14 in the morning, 1 h before the morning dose of saline/HB-EGF. Mice were killed at P18. b-e, Representative ×40 confocal images of CCl⁺ cells (red) derived from PDGFαR-CreER; Z/EG (GFP⁺) (green) progenitors at P18. f-i, Cells derived from PDGFαR-expressing progenitors after hypoxia and after HB-EGF treatment belong to the oligodendrocyte lineage (Olig2⁺). Representative ×40 confocal images of the subcortical white matter in all four groups at P18. In serial sections from each PDGFαR-CreER; Z/EG mouse, all GFP⁺ cells in the subcortical white matter co-stained with anti-Olig2⁺ antibody in all four groups (n=4 for each group). In all four groups, no GFP⁺ cells co-stained with anti-GFAP or anti-glutamate/aspartate transporter (GLAST) antibody (data not shown). j, Hypoxia results in a significant increase in the number of GFP⁺ cells in the white matter and HB-EGF has an additive effect (n=4 mice per group; one-way ANOVA, Bonferroni post-hoc test for individual comparisons). Histograms are presented as means±s.e.m. b-i, Scale bars, 50 μm. *P<0.05; **P<0.01.

FIG. 11 (Extended Data FIG. 7): HB-EGF treatment prevents hypoxia-induced changes in white matter axonal g ratios at P30. a, b, Scatter plots depicting g ratios versus axon diameter. The lines represent linear fits to pooled data from all mice for each genotype (n=3 mice per group). a, The scatter plot demonstrates that the Nx saline and Nx HB-EGF groups were similar. b, The scatter plot demonstrates that the Nx HB-EGF and Hyp HB-EGF groups were similar. c, Histogram demonstrating that, at P30, the percentage of myelinated subcortical white matter fibres was significantly decreased in the Hyp saline group. No significant different was found in the Hyp HB-EGF group. *P<0.05; **P<0.01.

FIG. 12 (Extended Data FIG. 8): Intranasal HB-EGF prevents loss of NAA after hypoxia. a, ¹H-NMR spectroscopy was performed on dissected white matter at P11, P18 and P30. A full-scale representative spectra is shown where the peak for NAA is at 2.0 p.p.m. The spectra shown on FIG. 4l is truncated. b, Western blots of aspartoacylase (ASPA), an enzyme found in oligodendrocytes and responsible for hydrolysation of NAA for myelin production in the developing brain. Hypoxia does not result in any significant change in the amount of ASPA present in the white matter at each of the time points listed (P11 and P30, n=4 for each group and age; P18, n=5 for each group; one-way ANOVA, Bonferroni post-hoc test for individual comparisons, except P11 unpaired t-test). Histograms are presented as means±s.e.m.

FIG. 13 (Extended Data FIG. 9): Protocol for late HB-EGF administration used for this study. In this study, HB-EGF or saline was administered at a later time point. Beginning in the morning of P18, HB-EGF or saline was administered every 12 h until the morning of P21. Intraperitoneal BrdU was administered 1 h before HB-EGF or saline administration, from P18-P21. The inclined beam-walking task was performed at P35. Only Rep mice were used for this study. The histogram is presented in FIG. 4p.

FIG. 14 (Extended Data FIG. 10): Intranasal HB-EGF accelerates oligodendrocyte maturation in the white matter after hypoxia by preventing Notch activation. a, Microdissected white matter was probed for activated Notch intracellular domain (NICD) and its ligand Delta1. Western blot analysis obtained from microdissected white matter at P11, P14.5 and P18 with actin as a loading control. b, c, Histograms represent quantification of the density of NICD (b) and Delta1 (c) signal normalized to actin. b, At P11 and P14.5, there was a significant increase in the amount of NICD in the Hyp group. No significant difference was evident at P18. The Hyp HB-EGF group had no significant increase at P14.5 compared with the Nx HB-EGF group, and significantly less than the Hyp saline group (n=4 mice for each group and age; one-way ANOVA, Bonferroni post-hoc test for individual comparisons). c, Delta1 was increased at P14.5 only in the Hyp saline group (P11: n=4 for each group; P14.5 and P18: n=5 for each group and age; one-way ANOVA, Bonferroni post-hoc test for individual comparisons). d-g, Representative ×40 confocal images of subcortical white matter in the transgenic Notch reporter (TNR) mice, where eGFP is expressed upon activation of Notch effector C-promoter binding factor 1 (CBF1), a downstream transcriptional target of Notch. h, Histogram represents the number of eGFP⁺Olig2⁺ cells at P14.5 in the white mater. The Hyp saline group showed a significant increase in eGFP⁺Olig2⁺ cells, corresponding to enhanced Notch activation in oligodendrocyte lineage cells. This contributes to delayed maturation of oligodendrocyte lineage cells observed after hypoxia (n=4 mice per group; one-way ANOVA, Bonferroni post-hoc test for individual comparisons). Histogram is presented as means±s.e.m. d-g, Scale bars, 50 μm. *P<0.05; **P<0.01.

### DETAILED DESCRIPTION OF THE INVENTION

Agonists of the Epidermal Growth Factor Receptor (“EGFR”) may be used in the invention. These include EGF, HB-EGF and other known EGFR agonists. Known EGFR (Erb-1) agonists include HB-EGF, EGF, TGF-α, amphiregulin, beta cellulin, epigen, and epiregulin.

The amino acid sequence of Epidermal Growth Factor (‘EGF”) is known and is given by Accession No. AAS83395, version AAS83395.1; GI:46242544 (SEQ ID NO: 1).

The amino acid sequence of heparin-binding EGF-like growth factor is known and is given by Accession No. NP_001936, version NP_001936.1 or by GI:450341 (SEQ ID NO: 2).

Variants of EGF or heparin-binding EGR-like growth factor may also be employed such as polypeptides having at least 70%, 85%, 90%, 95%, 96%, 97%, 98% or 99% identity or similarity to EGF or to heparin-binding EGF-like growth factor described herein and preferably exhibiting substantially the same functions. BLASTP is used to identify an amino acid sequence having at least 70%, 75%, 80%, 85%, 87.5%, 90%, 92.5%, 95%, 97.5%, 98%, 99% sequence similarity to a reference amino acid sequence of SEQ ID NO: 1 or 2 using a similarity matrix such as BLOSUM45, BLOSUM62 or BLOSUM80. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity or similarity to the amino acid sequences disclosed herein are contemplated and encompassed by this disclosure. Polypeptides comprising portion or fragments of the amino acid sequence of SEQ ID NO: 1 or 2 or variants thereof are also contemplated. The variants, portions or fragments of the amino acid sequence of SEQ ID NO: 1 or 2 preferably share at least one functional activity of the EGF of SEQ ID NO: 1 or heparin-binding EGF-like growth factor of SEQ ID NO: 2, especially the ability to prevent or treat neonatal brain injury such as white matter injury.

The analogs, variants and modified forms of the EGF of SEQ ID NO: 1 and the EGF-like growth factor of SEQ ID NO: 2 can be produced by techniques well-known in the molecular biological, biochemical and chemical arts. For example, they can be produced by expression of a polynucleotide or gene encoding these products in a suitable host cell or other techniques described by and incorporated by reference to Green & Sambrook, Molecular Cloning: A Laboratory Manual, Fourth Edition (2012). A DNA construct or expression vector for this purpose may be produced by conventional recombinant DNA techniques, such as by site-directed mutagenesis a sequence encoding SEQ ID NO: 1 or 2. The polynucleotide sequences that encode the amino acid sequence of SEQ ID NO: 1 or 2 or portions, fragments or polypeptide variants of SEQ ID NO: 1 or 2 are described by reverse translating the protein sequence using the genetic code and may be obtained by conventional means, such as by chemical synthesis or by recombinant amplification or expression. Such polynucleotides may be incorporated into vectors or DNA constructs, such as into expression vectors that express a heparin-binding EGF-like growth factor polypeptide when transformed into a cell.

Alternatively, such products may be produced in whole or part chemical synthesis such as by a Merrifield-type synthesis. Chemical synthesis is preferred for variants, analogs or modified forms that contain non-naturally-The functional activities of such engineered products can be tested or screened for functional activity by conventional methods including the assays and tests disclosed herein. Such variants, analogs and modified forms of EGF or HB-EGF may be incorporated into pharmaceutical compositions, for example, by admixture with a sterile physiologically acceptable carrier or excipient to prepare a pharmaceutically acceptable composition. The particular ingredients and form of such a composition can be chosen depending on the route and site of administration.

Suitable pharmaceutically acceptable carriers include but are not limited to water, salt solutions (e.g., normal NaCl/saline), buffered saline, alcohols, glycerol, ethanol, gum arabic, vegetable oils, benzyl alcohols, polyethylene glycols, gelatin, carbohydrates such as lactose, amylase or starch, dextrose, magnesium stearate, talc, silicic acid, viscous paraffin, fatty acid esters, hydroxymethylcellulose, polyvinyl pyrrolidone, etc., as well as combinations thereof. In addition, carriers such as liposomes and microemulsions may be used. The heparin-binding EGF-like growth factor, a functional fragment thereof, or a variant thereof may also be covalently attached to a protein carrier such as albumin, or a polymer, such as polyethylene glycol so as to modulate pharmacokinetics such as to prolong biological half-life. Auxiliary agents, such as lubricants, preservatives, stabilizers, wetting agents, emulsifiers, salts for influencing osmotic pressure, buffers, coloring, flavoring and/or aromatic substances and the like that do not deleteriously react with heparin-binding EGF-like growth factor, a functional fragment thereof, or a variant thereof may be included.

The heparin-binding EGF-like growth factor, a functional fragment thereof, or a variant thereof used in the methods described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, and the like.

A therapeutically effective amount of the heparin-binding EGF-like growth factor, a functional fragment thereof, or a variant thereof for the treatment of a particular patient having a disorder or condition will depend on the nature of the disorder or condition, and can be determined by standard clinical techniques. In vitro or in vivo assays may be employed to identify optimal dosage ranges. The precise dose to be employed in the formulation will often depend on the route of administration and the severity of the symptoms of the disease or condition, and should be decided according to the judgment of a practitioner and each patient's circumstances. Effective doses may be extrapolated from dose-response curves derived from in vitro or animal model test systems.

The heparin-binding EGF-like growth factor, a functional fragment thereof, or a variant thereof used in the methods described herein may be covalently or non-covalently modified to facilitate their transfer across the blood-brain barrier. Similarly, these agonists may be admixed with solutions, carriers or excipients to facilitate their transfer through or behind the blood-brain barrier.

The inventors examined whether enhanced EGFR signalling stimulates the endogenous response of EGFR-expressing progenitor cells during a critical period after brain injury, and promotes cellular and behavioural recovery in the developing brain. Using an established mouse model of very preterm brain injury, the inventors demonstrate that selective overexpression of human EGFR in oligodendrocyte lineage cells or the administration of intranasal heparin-binding EGF immediately after injury decreases oligodendroglia death, enhances generation of new oligodendrocytes from progenitor cells and promotes functional recovery. Furthermore, these interventions diminish ultrastructural abnormalities and alleviate behavioural deficits on white-matter-specific paradigms. Inhibition of EGFR signalling with a molecularly targeted agent used for cancer therapy demonstrates that EGFR activation is an important contributor to oligodendrocyte regeneration and functional recovery after DWMI. The results shown herein provide direct evidence that targeting EGFR in oligodendrocyte progenitor cells at a specific time after injury is a clinically feasible treatment of neonates, especially premature children, with white matter injury.

Chronic neonatal hypoxia is a clinically relevant model of premature brain injury caused by insufficient gas exchange from poor lung development⁵. This ‘hypoxic’ state is a major contributor to DWMI—a common finding in infants born very preterm (VPT)—resulting in sensorimotor deficits that persist throughout their lifetime^{1,2,6}. A mouse model of chronic hypoxia was used, which replicates DWMI and other neuropathological hallmarks of brain injury resulting from premature birth⁷⁻⁹.

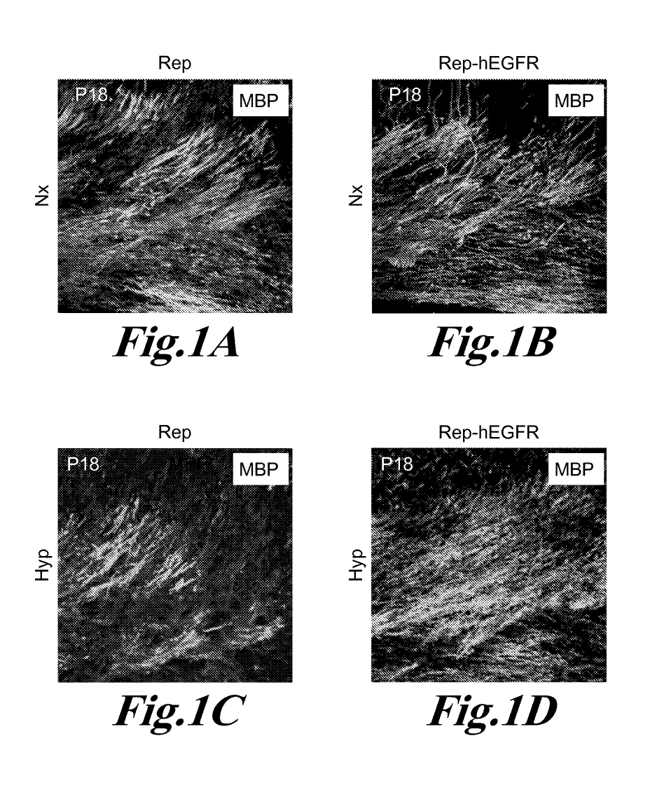

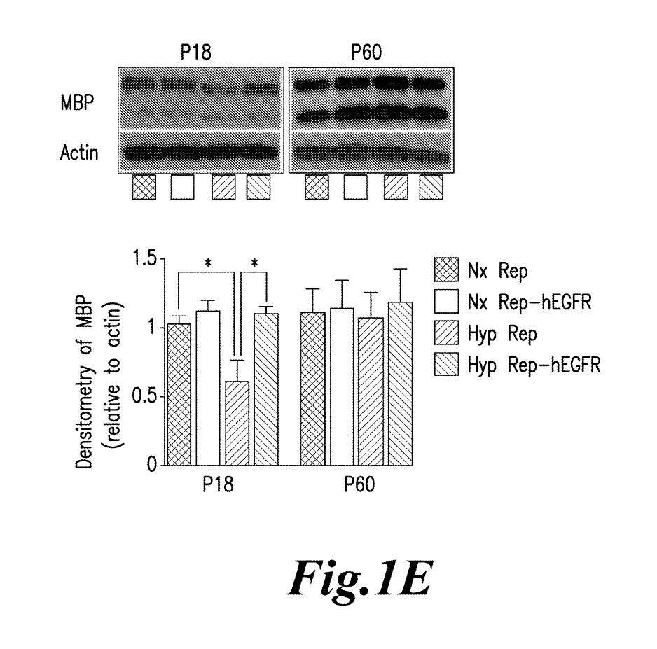

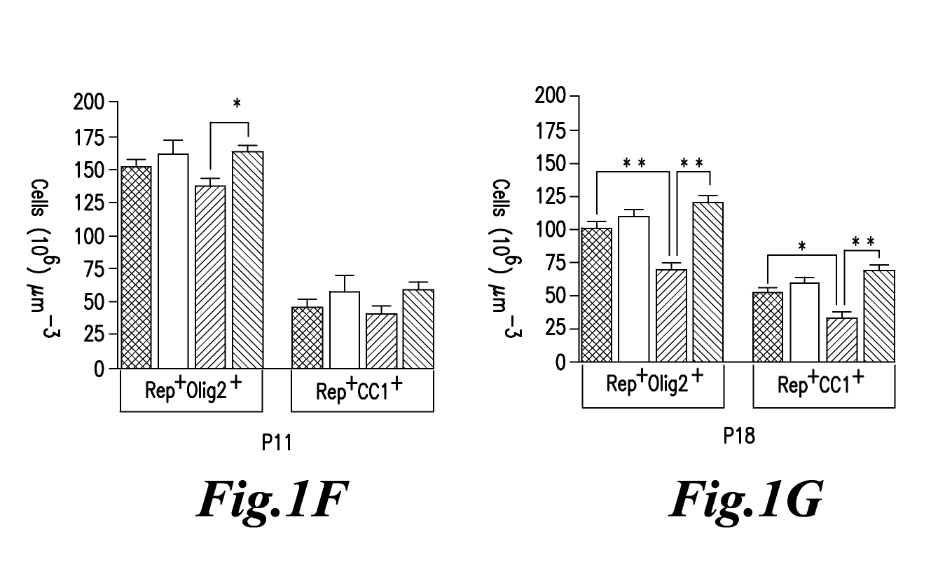

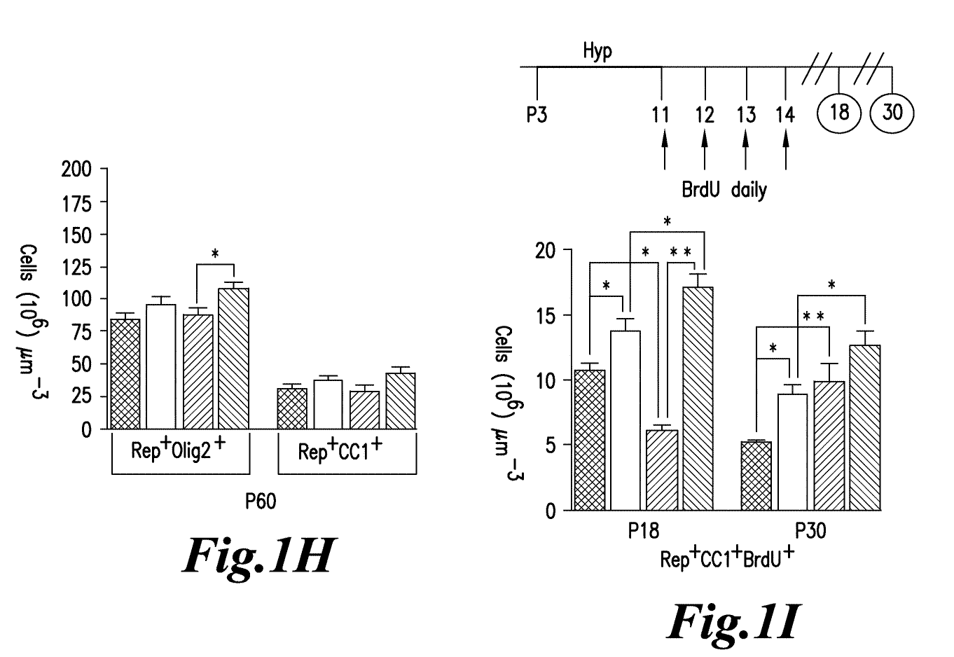

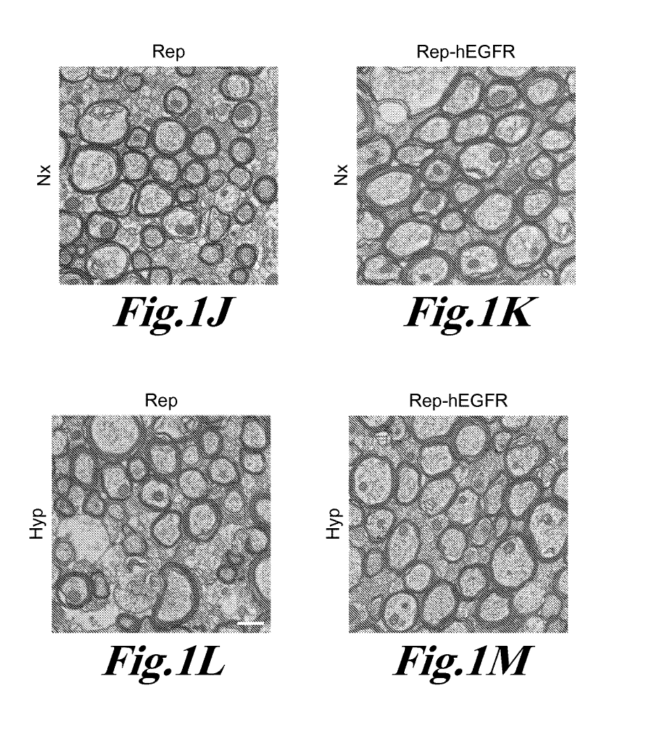

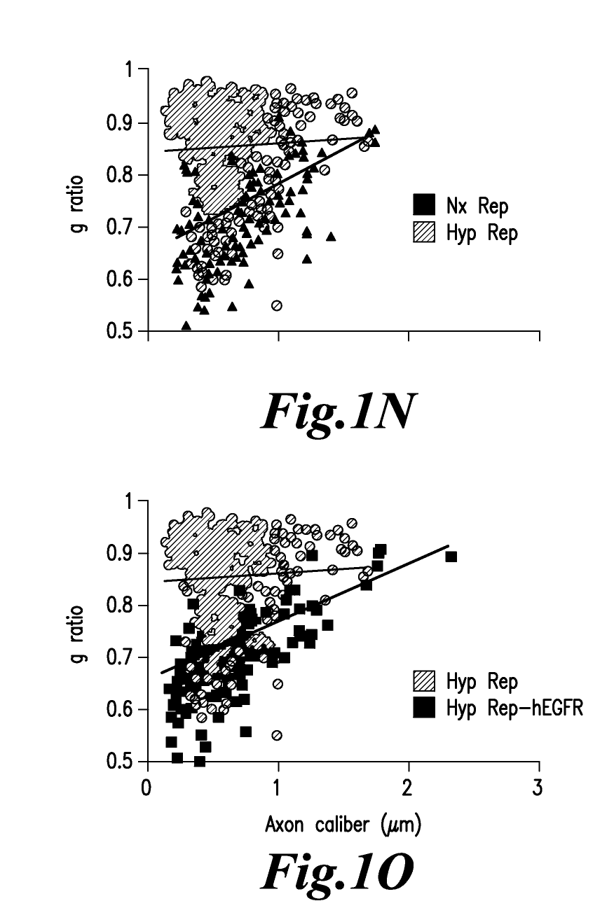

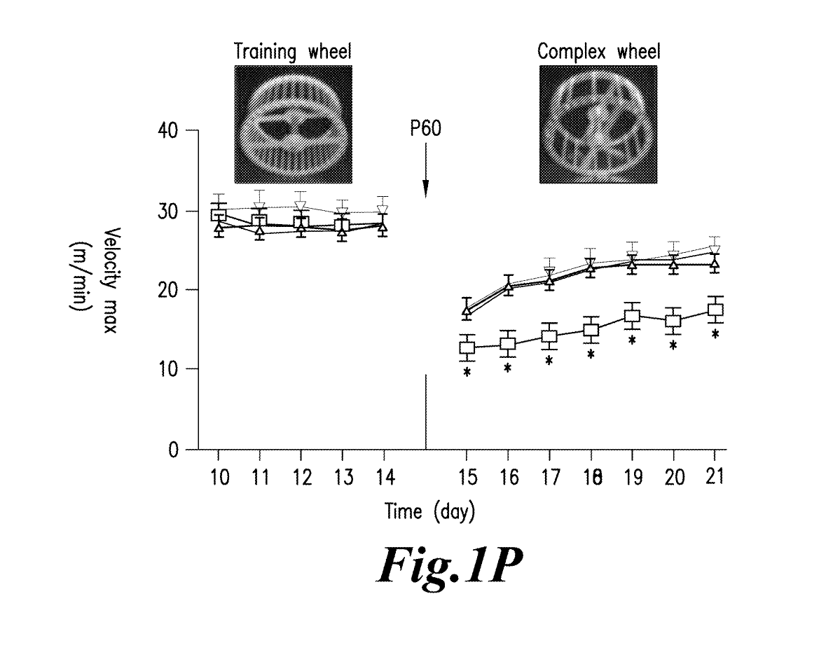

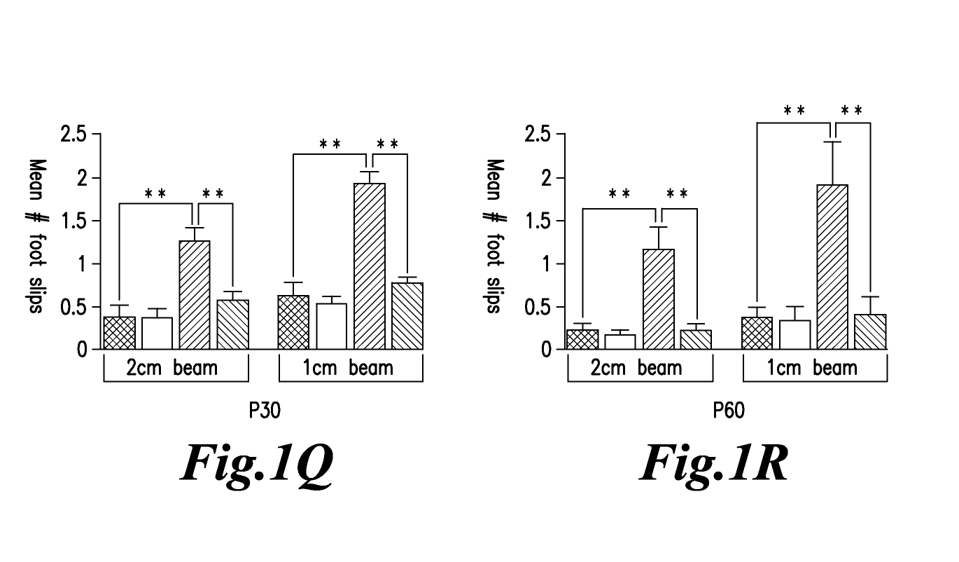

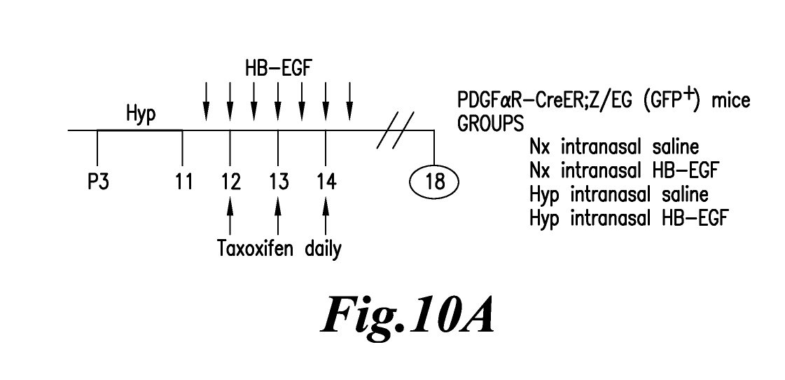

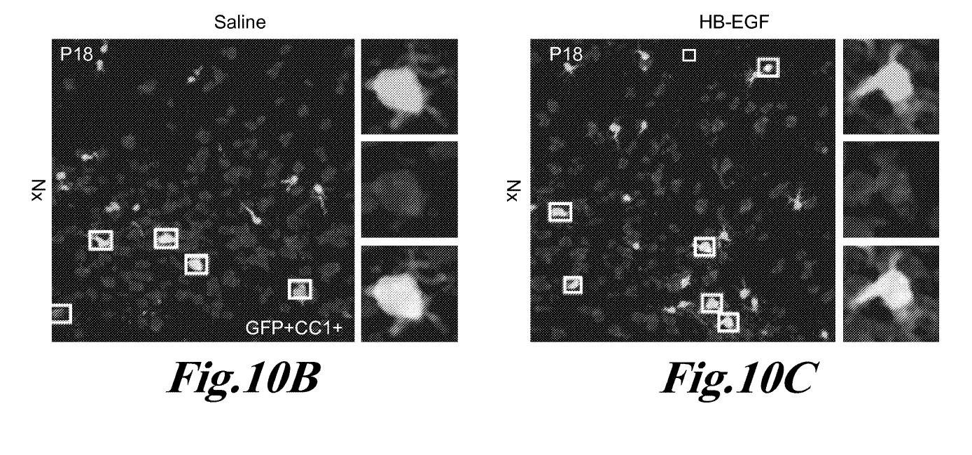

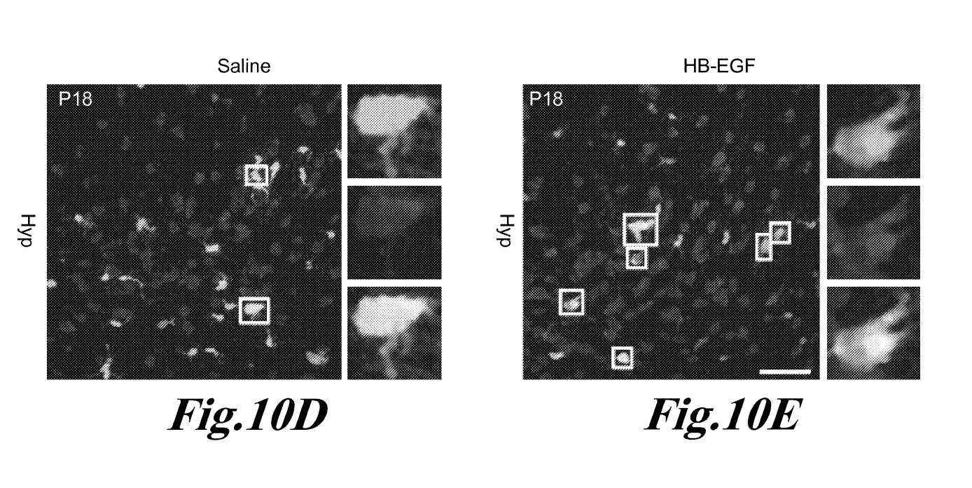

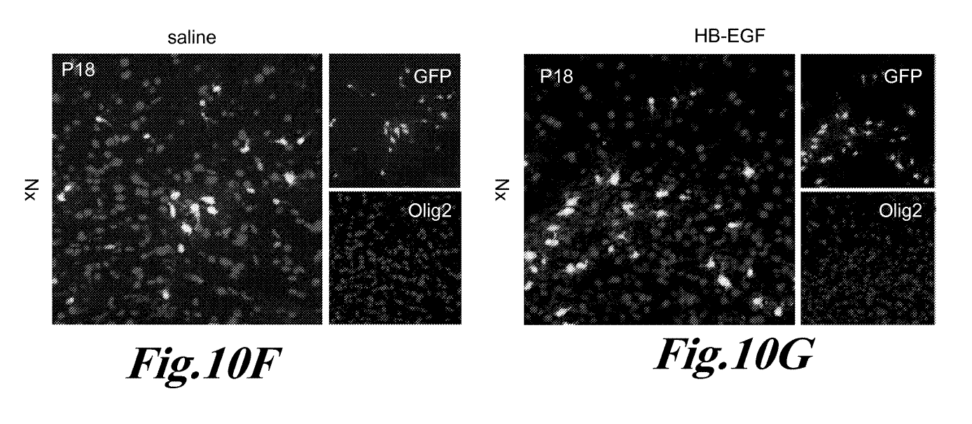

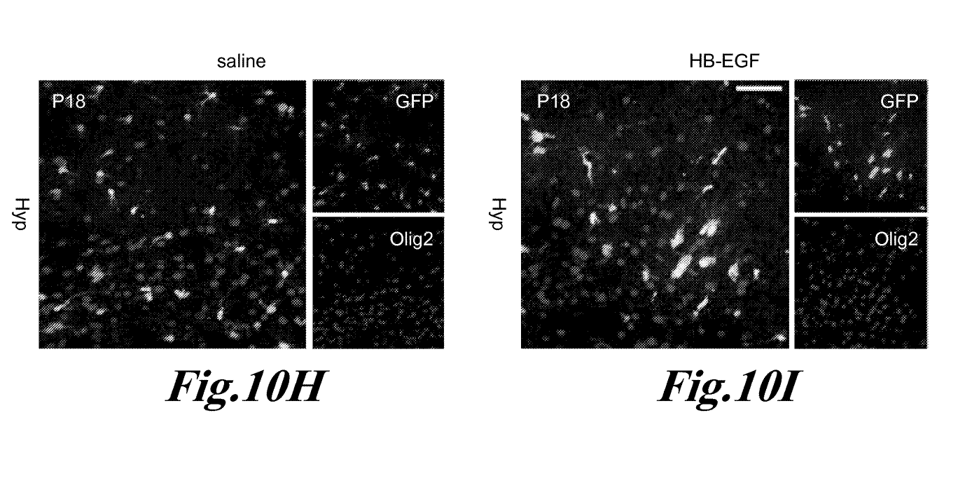

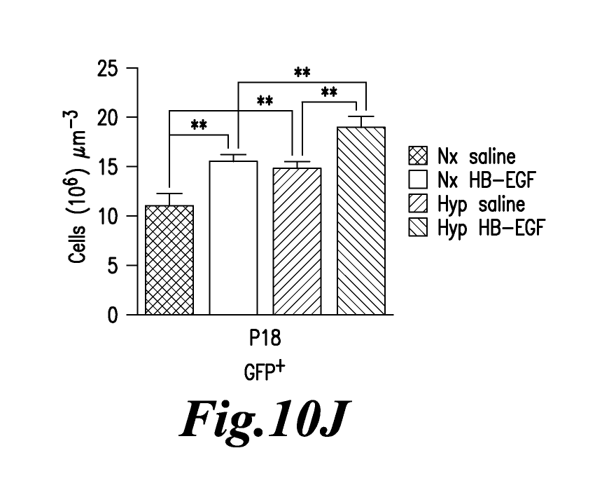

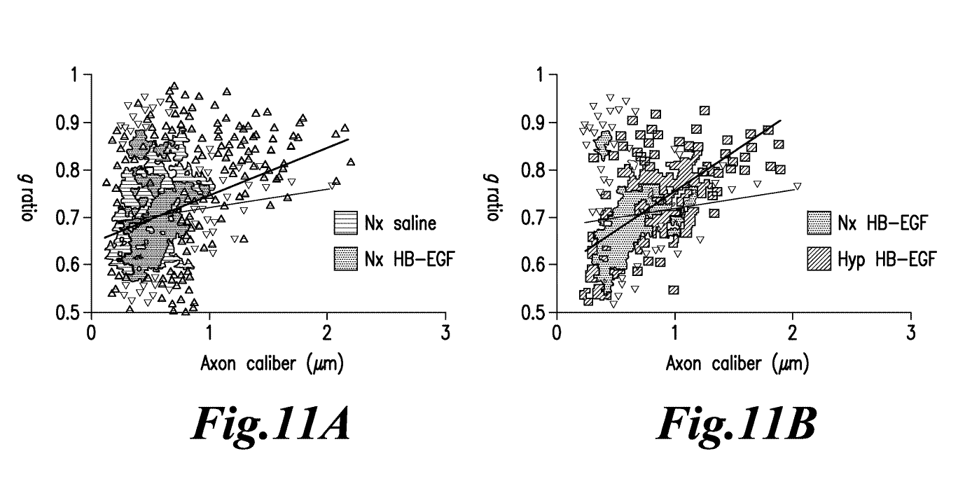

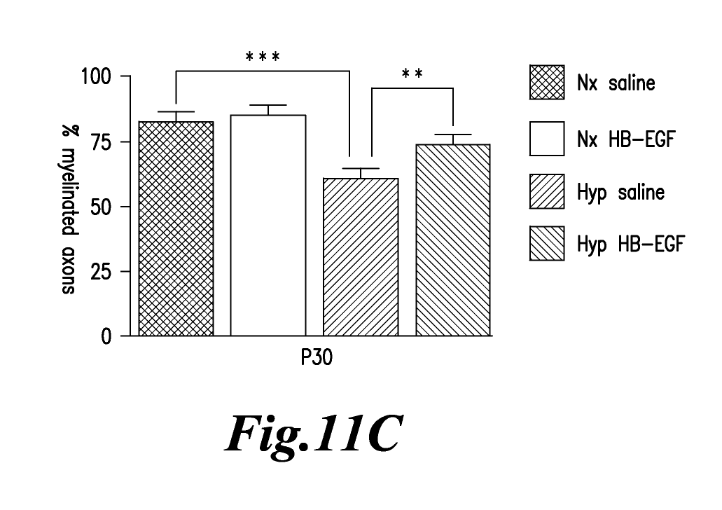

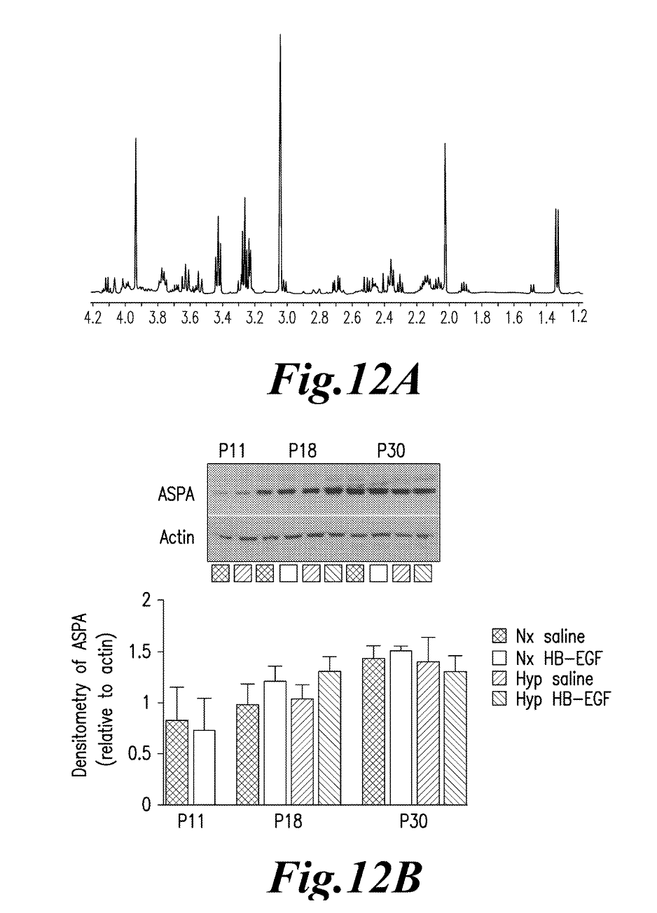

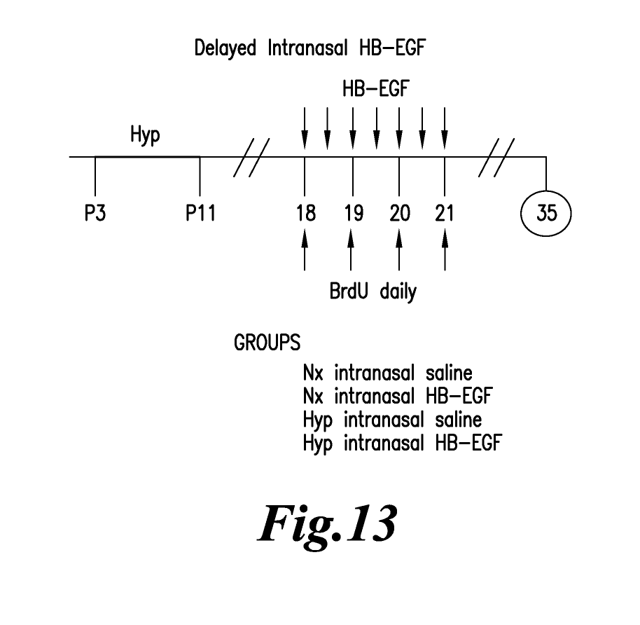

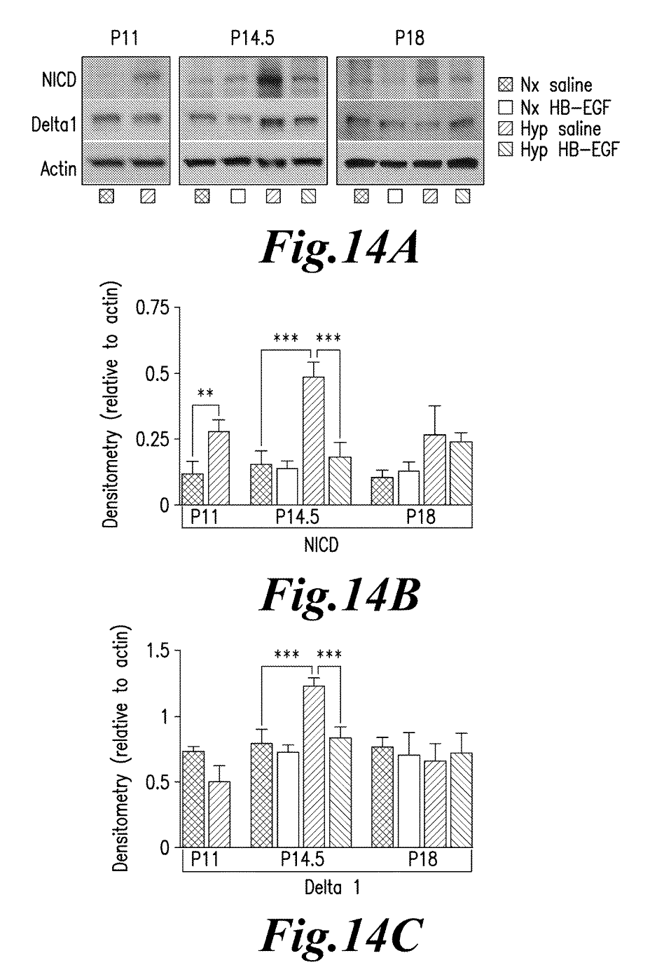

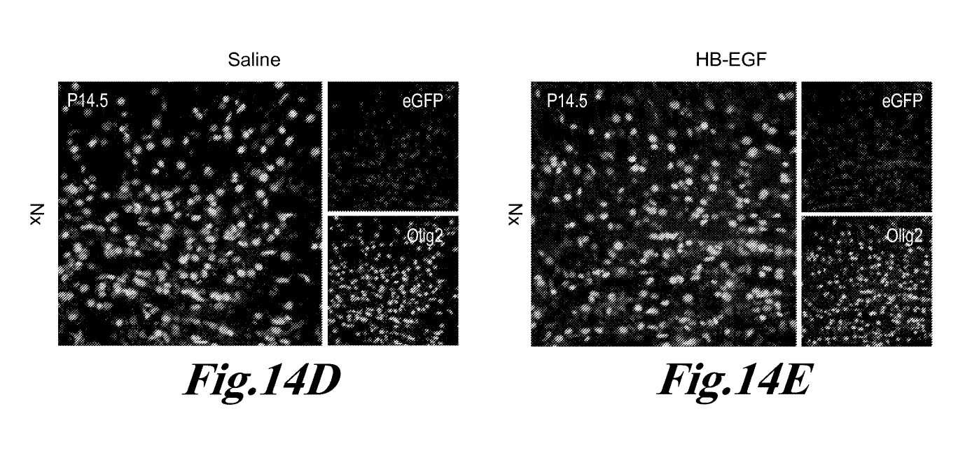

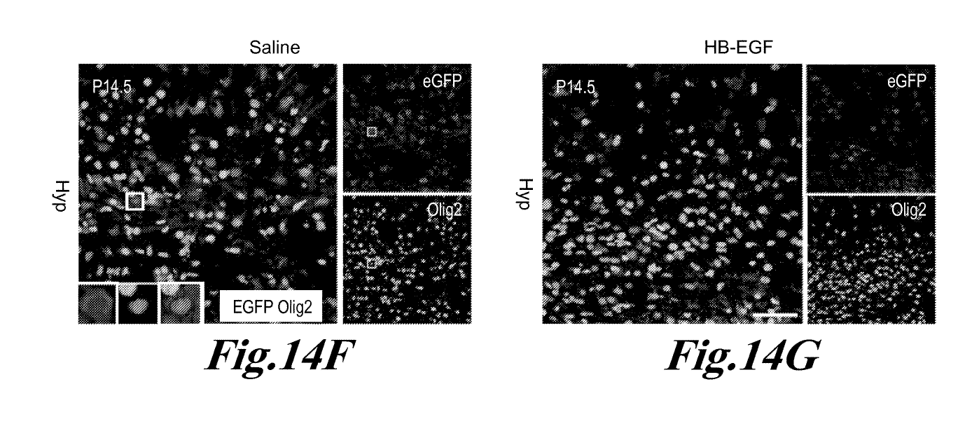

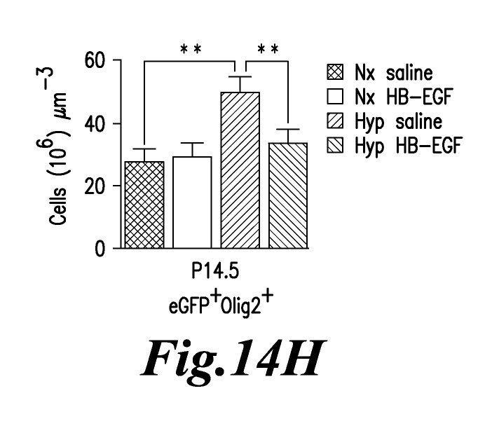

The cellular and molecular mechanisms underlying DWMI in VPT children—and in chronic hypoxia—are unknown. It has been previously demonstrated that enhanced EGFR signalling in white matter oligodendrocyte lineage cells promotes their proliferation, migration, myelination and remyelination in the adult^{4,10}. The inventors observed a significant increase in endogenous EGF levels in the white matter after chronic hypoxia (Extended Data FIG. 1). Therefore, we compared oligodendrocyte development in white matter injury and recovery in 2′,3′-cyclic nucleotide 3′-phosphodiesterase (CNP) enhanced green fluorescent protein (eGFP) mice (Rep mice) and Rep mice in which human (h)EGFR was overexpressed in the oligodendrocyte lineage under the CNP promoter (Rep-hEGFR mice)^{4,11-13}. Hypoxia decreased myelin basic protein (MBP) expression in the white matter of Rep mice, but not in Rep-hEGFR mice (FIG. 1a-e). At postnatal day (P)60, MBP expression recovered in the hypoxia-treated Rep group (FIG. 1e). At P11, chronic hypoxia did not cause any change in the number of Rep⁺Olig2⁺ cells and mature (Rep⁺CCl⁺) oligodendrocytes (FIG. 10. At P18, we observed a decrease in Rep⁺Olig2⁺ and Rep⁺CCl⁺ oligodendrocytes in the white matter of hypoxia-treated Rep mice (FIG. 1g), but no change in the Rep-hEGFR mice. Oligodendrocyte recovery was evident by P60 in the hypoxia-treated Rep group (FIG. 1h).

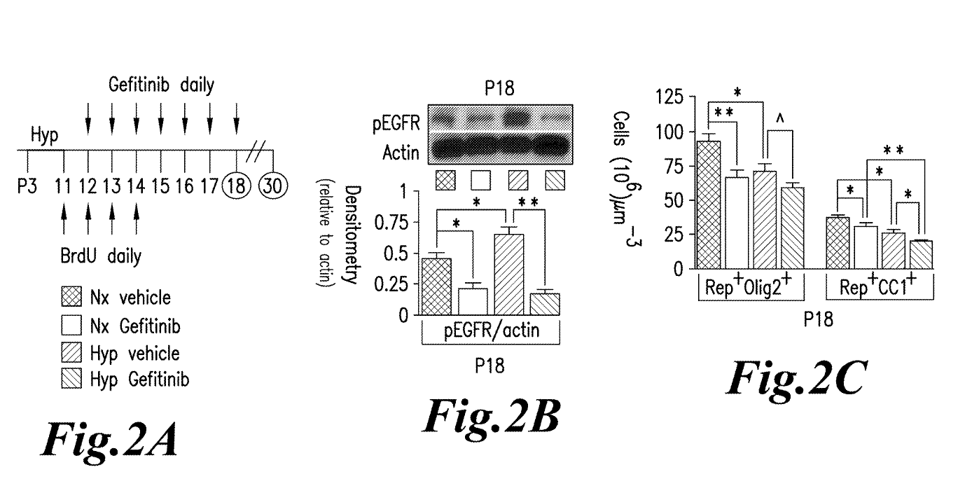

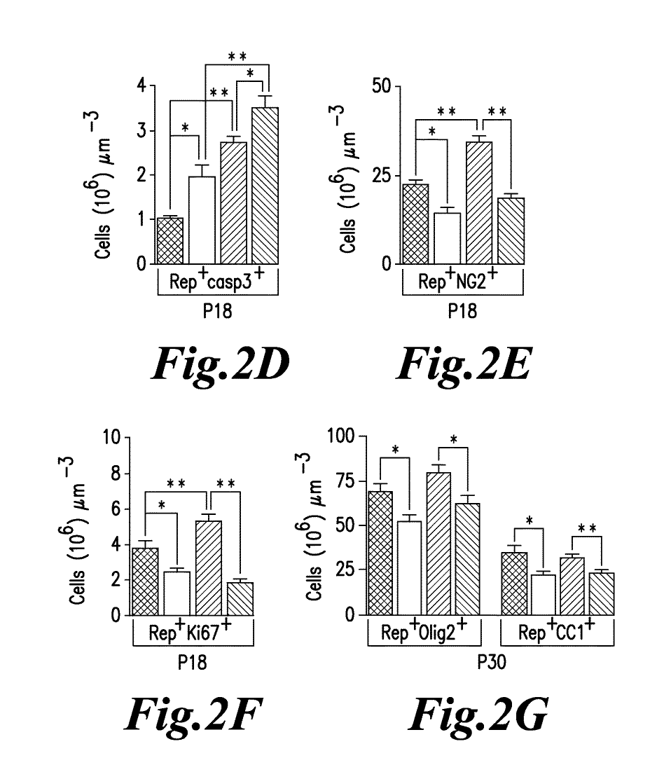

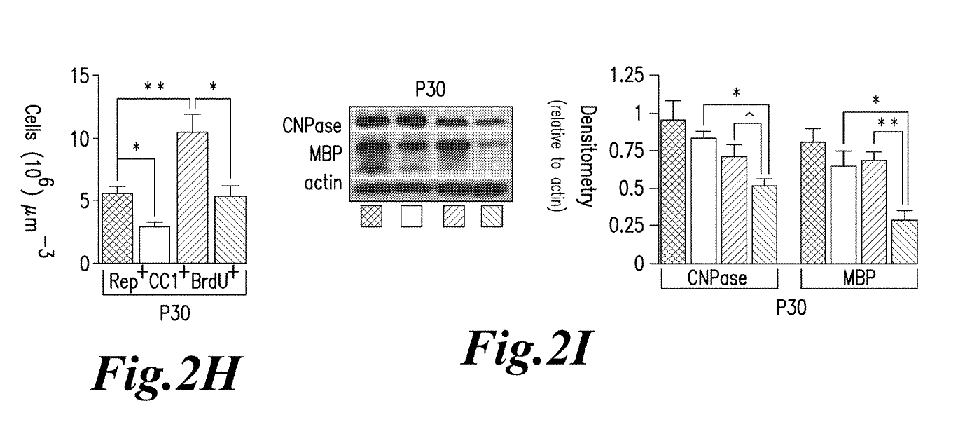

There was an increase in apoptosis of oligodendrocyte lineage cells in Rep mice after hypoxia treatment at P11 and P18, but no change at P60 (Extended Data FIG. 2e). No significant apoptosis was observed in Rep-hEGFR mice (Extended Data FIG. 2e). Hypoxia caused an increase in the number of propidium iodide (PI)⁺ cells (data not shown) and Rep⁺PI⁺ cells (Extended Data FIG. 2a-d, f). This increase was not observed in Rep-hEGFR mice. These results indicate that enhanced EGFR expression prevents oligodendrocyte loss by decreasing cell death after exposure to chronic hypoxia.

We next assessed the effects of hypoxia on oligodendrocyte progenitor (Rep⁺NG2⁺) cells (OPCs) in the white matter (Extended Data FIG. 2g-k). Enhanced hEGFR expression caused an increase in Rep⁺NG2⁺ OPCs at P11 and P18 (normoxia Rep mice versus normoxia Rep-hEGFR mice; Extended Data FIG. 2k). Hypoxia caused a significant increase in white mater OPCs in both Rep and Rep-hEGFR mice at the same ages (Extended Data FIG. 2k). Similar findings were obtained after assessing proliferation of Rep⁺ oligodendrocyte lineage cells (Extended Data FIG. 2l). Enhanced hEGFR expression increased Rep⁺NG2⁺ OPC proliferation in the normoxia group, and had an additive effect on hypoxia-induced OPC proliferation (Extended Data FIG. 2m). Enhanced hEGFR expression increased oligodendrogenesis at P18, but, at P30, no difference was evident between hypoxia-treated Rep and hypoxia-treated Rep-hEGFR mice (FIG. 1i). These results indicate that enhanced hEGFR expression in oligodendrocytes promoted the generation of new oligodendrocytes after chronic hypoxia.

We used electron microscopy to determine whether chronic hypoxia caused myelination abnormalities, and to assess whether EGFR overexpression rescued these abnormalities. (FIG. 1j-o). At P60, when oligodendrocyte cell numbers and MBP expression recovered, myelination was still abnormal after hypoxia (FIG. 1j-o). Hypoxia caused a significant increase in the g ratio and hEGFR expression prevented this increase (FIG. 1n, o).

Next, we investigated behavioural deficits resulting from DWMI after perinatal chronic hypoxia by using subcortical white-matter-dependent sensorimotor behavioural tests (complex wheel and inclined beam-walking task)¹⁴⁻¹⁸. In the complex wheel task, there was no difference in training maximum velocity (Vₘₐₓ) between all four groups (FIG. 1p). On day 15, all four groups had a decline in Vₘₐₓ; however, the largest decline was in the hypoxia-treated Rep group (FIG. 1p). The hypoxia-treated Rep group performed poorly on the complex wheel (days 15-21), as compared to the other three groups (FIG. 1p), suggesting altered subcortical white matter integrity.

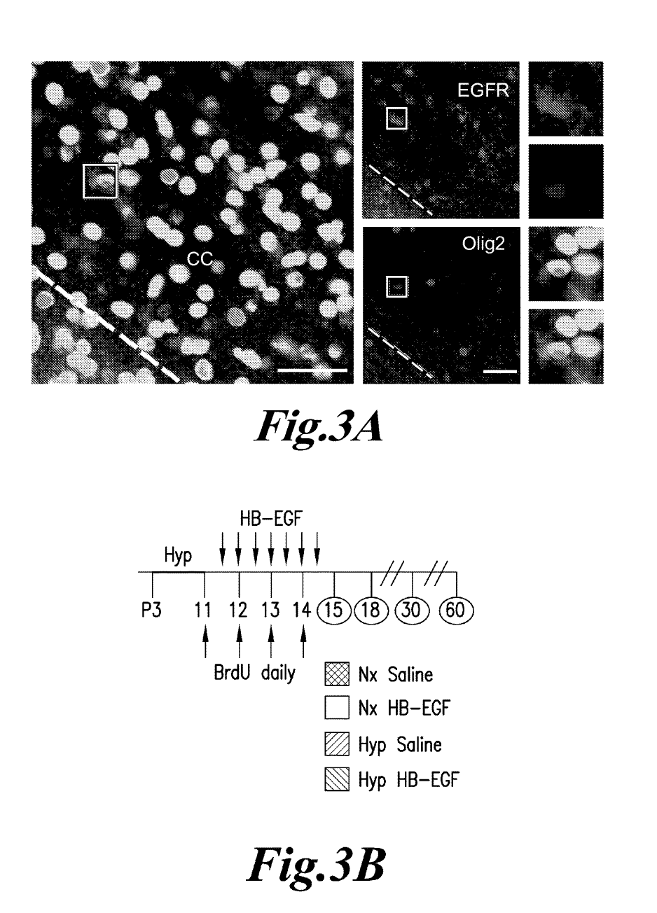

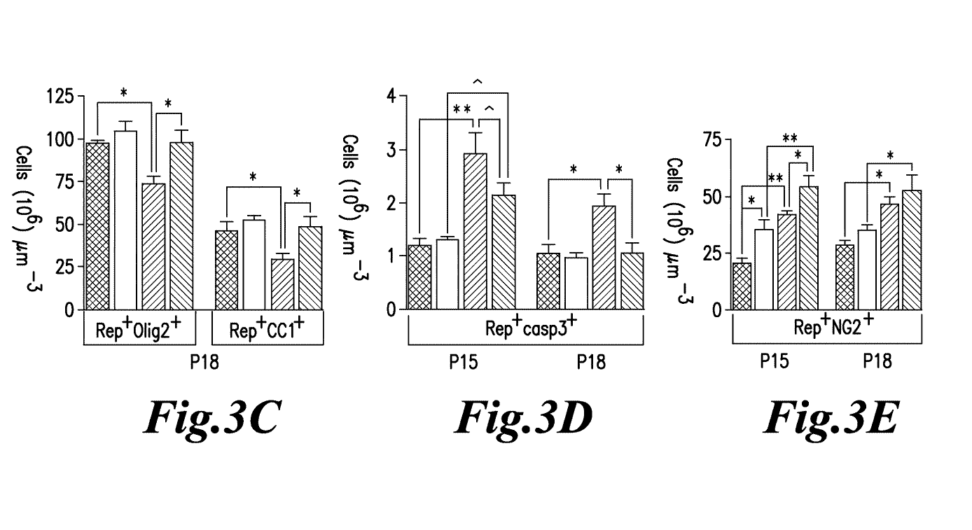

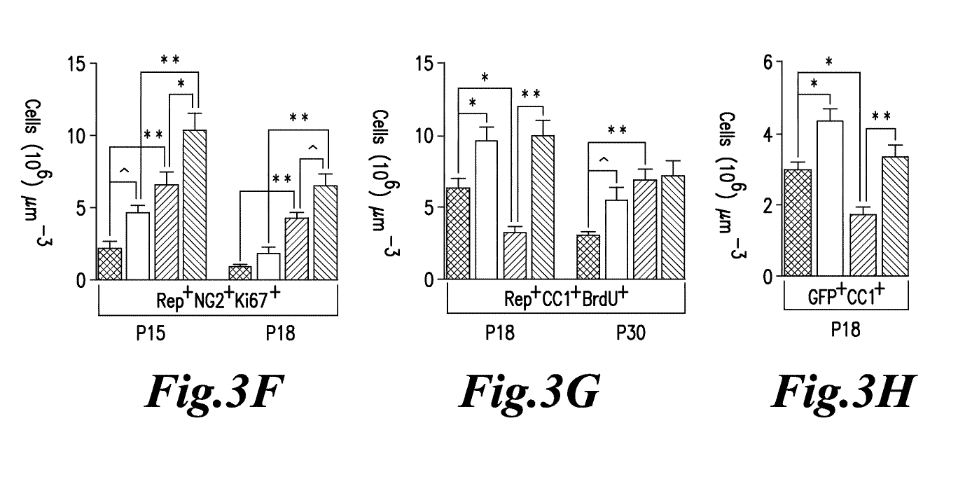

The inclined beam-walking task^{17,18} requires subcortical white matter integrity (Extended Data FIG. 3a-c) and no difference was observed between Rep mice and C57BL/6 mice (Extended Data FIG. 3d). At P30, the hypoxia-treated Rep group displayed more foot slips than the normoxia Rep group (FIG. 1q). Hypoxia-treated Rep-hEGFR mice showed no significant increase in the number of foot slips as compared with the normoxia Rep-hEGFR group. At P60, the hypoxia-treated Rep group continued to show an increase in number of foot slips, and no difference was seen in the hypoxia-treated Rep-hEGFR group (FIG. 1r). These behavioural studies confirm that exposure to chronic hypoxia during a critical period in myelin development results in poor performance on white-matter-specific behavioural tasks. Enhanced EGFR activity prevents the effects of hypoxia, strongly suggesting that EGFR signalling in oligodendrocyte lineage cells has a crucial role in white matter recovery after perinatal injury.

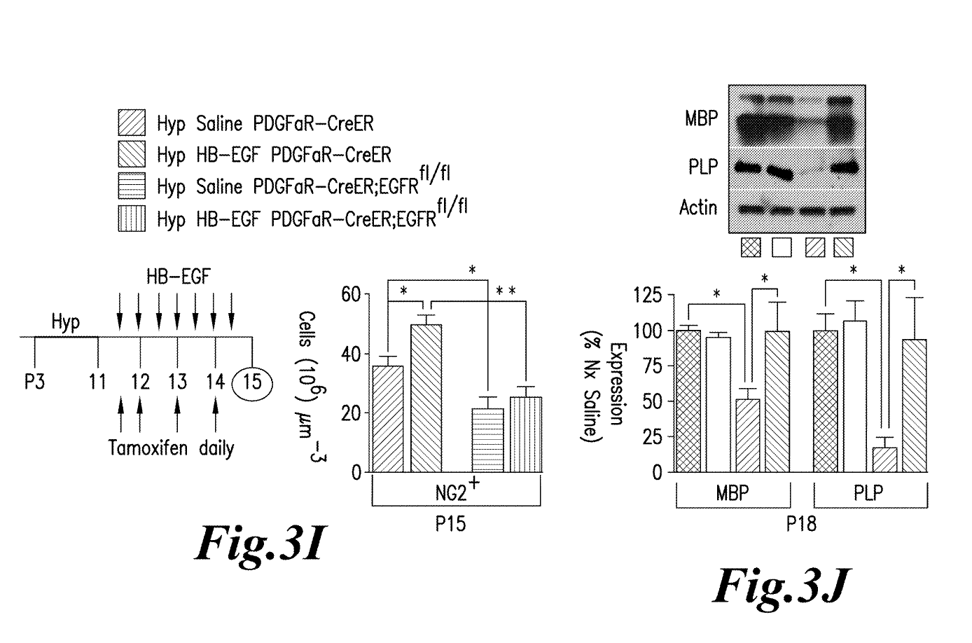

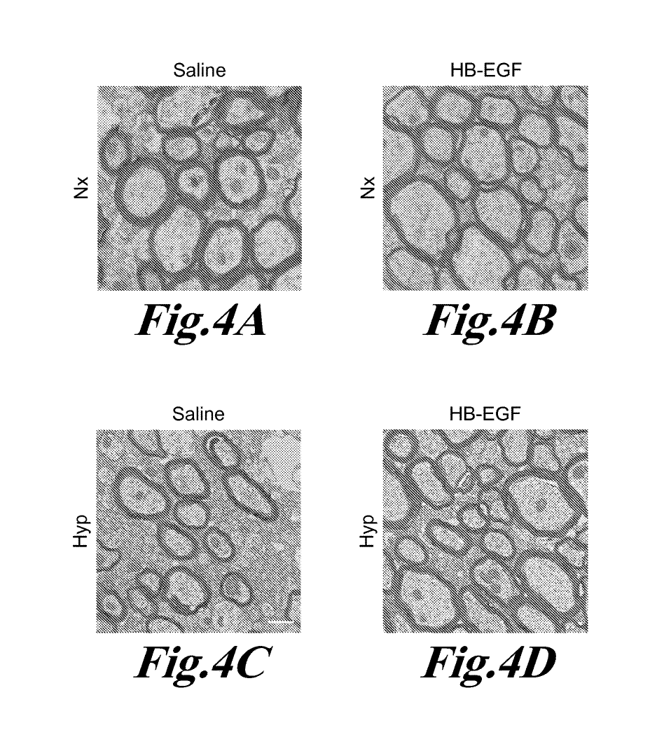

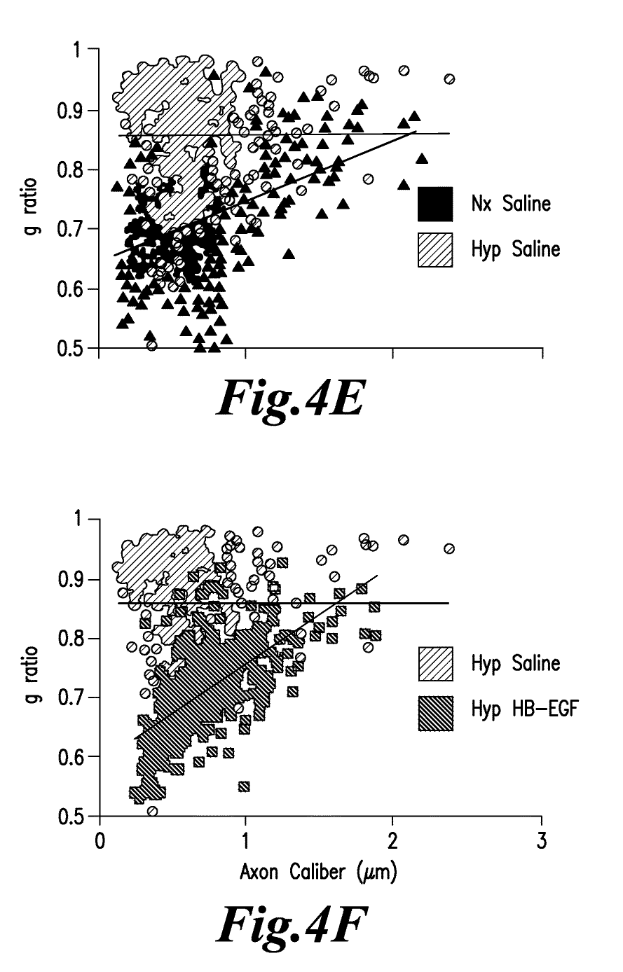

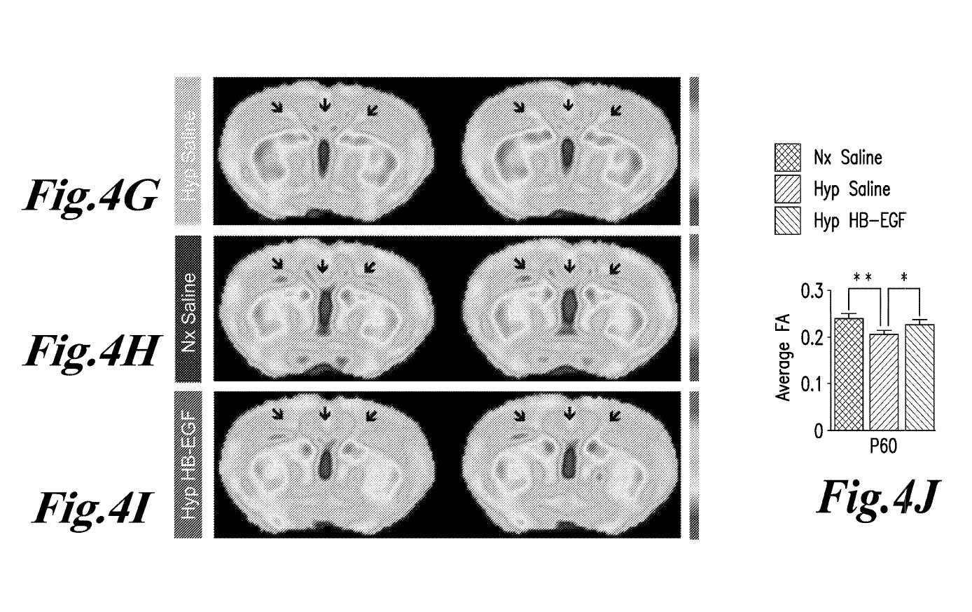

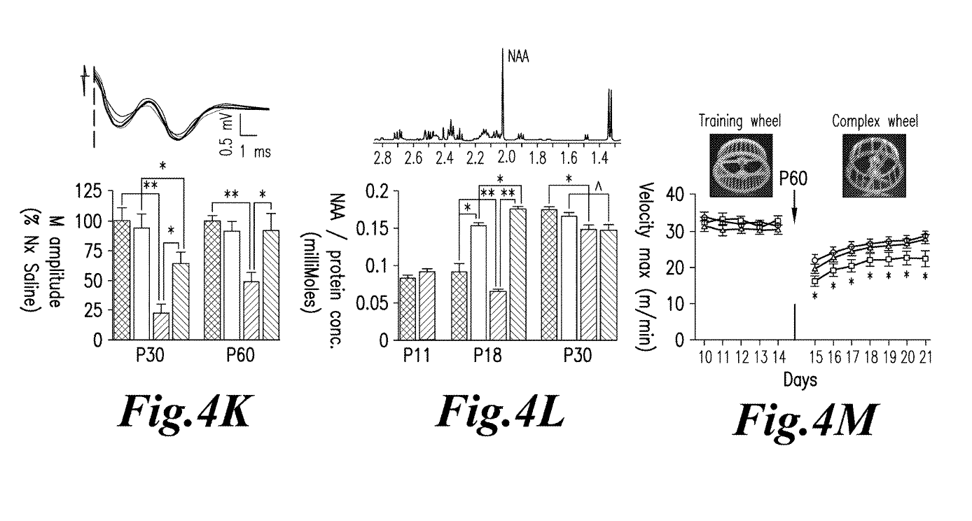

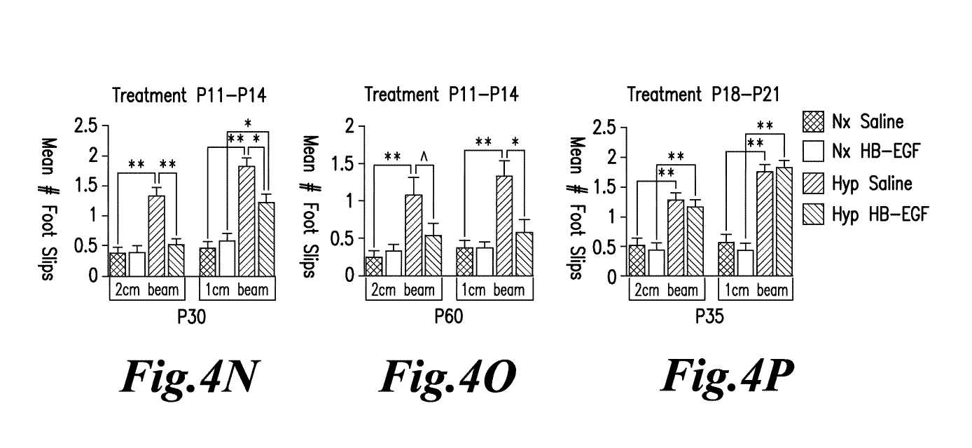

We directly tested the role of endogenous EGFR signalling in oligodendrocyte recovery after hypoxia by gefitinib administration from P12-P18 (FIG. 2a). Gefitinib, a specific EGFR antagonist¹⁹, caused a small reduction in basal phosphorylated (p)EGFR levels in the white matter of normoxia mice at P18, but completely prevented the increase in pEGFR induced by hypoxia (FIG. 2b). Gefitinib caused a decrease of white matter Rep⁺Olig2⁺ and Rep⁺CCl⁺ oligodendrocyte lineage cells in normoxia mice (FIG. 2c). Hypoxia resulted in a decrease of these cell populations, which were further reduced by gefitinib (FIG. 2c). This decrease was attributed to a significant increase in oligodendrocyte apoptosis in both normoxia and hypoxia groups (FIG. 2d). Gefitinib also decreased NG2⁺ OPCs in the white matter of normoxia mice, and prevented the increase observed after hypoxia (FIG. 2e). Similarly, gefitinib decreased white matter oligodendrocyte linage cell proliferation in normoxia mice and prevented the proliferative response observed after hypoxia (FIG. 2f). Gefitinib also decreased Sox2- and Ascl1-expressing progenitors (Extended Data FIG. 4a-j). At P30, gefitinib still caused a decrease in oligodendrocyte lineage cells and mature oligodendrocytes in normoxia mice, and prevented oligodendrocyte recovery and oligodendrogenesis observed after hypoxia (FIG. 2g, h). Finally, gefitinib prevented the recovery in CNPase and MBP protein levels observed after hypoxia at P30 (FIG. 2i). These results confirm that endogenous EGFR signalling is important in white matter cellular and biochemical recovery after hypoxia.

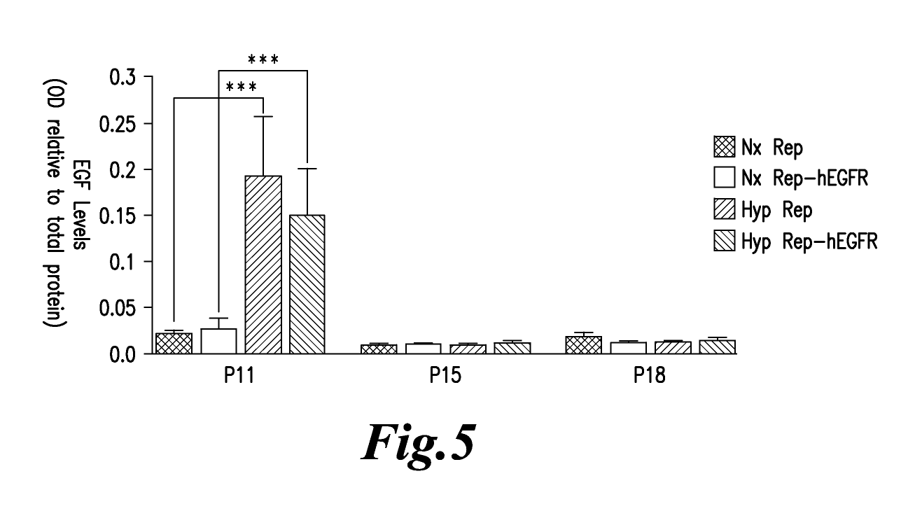

The inventors examined whether directly targeting EGFR with a selective ligand (recombinant heparin-binding EGF (HB-EGF)) delivered through the intranasal route promoted cellular recovery of white matter oligodendrocytes after hypoxia. The clinical relevance of targeting endogenous oligodendrocytes was demonstrated by the presence of EGFR-expressing oligodendrocyte lineage cells (Olig2⁺EGFR⁺) in neonatal preterm human white matter (FIG. 3a). The intranasal route allows rapid drug delivery directly to the brain from the nasal mucosa²⁰⁻²². HB-EGF via the intranasal route enters the brain^{20,23} and acts on the white matter (Extended Data FIG. 5a-c), where Rep⁺pEGFR⁺ cells could be identified (Extended Data FIG. 5d). FACS purification of hypoxia-exposed white matter Rep⁺ cells after HB-EGF treatment directly demonstrated activation of EGFR in oligodendrocyte lineage cells (Extended Data FIG. 5e). Treatment with seven doses of intranasal HB-EGF from P11-P14 (FIG. 3b) prevented white matter oligodendrocyte lineage cell loss after hypoxia (FIG. 3c). Rep⁺ cells were stained for caspase 3 in all groups. At all ages examined, HB-EGF treatment reduced (P15) or prevented (P18) the effects of hypoxia on oligodendrocyte cell death (FIG. 3d).

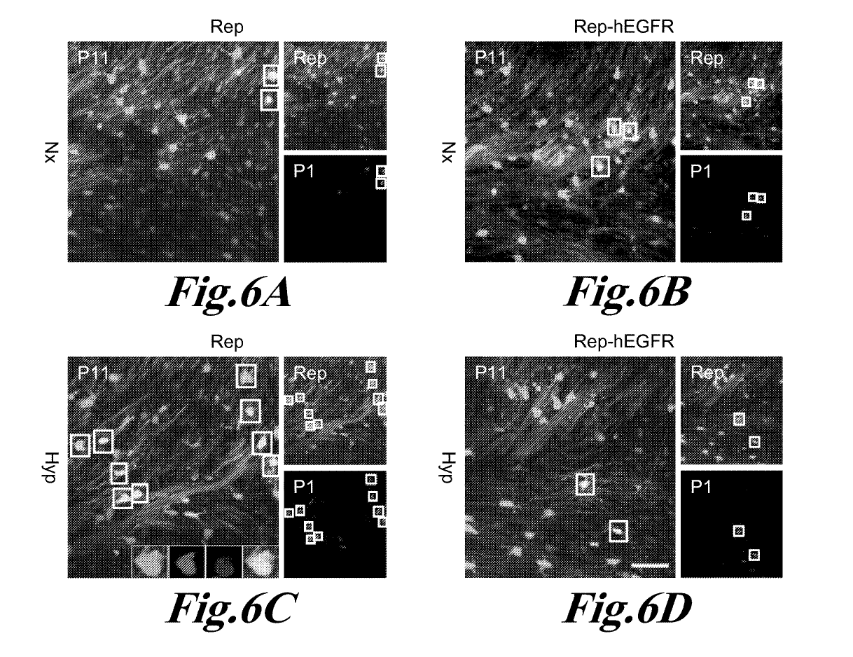

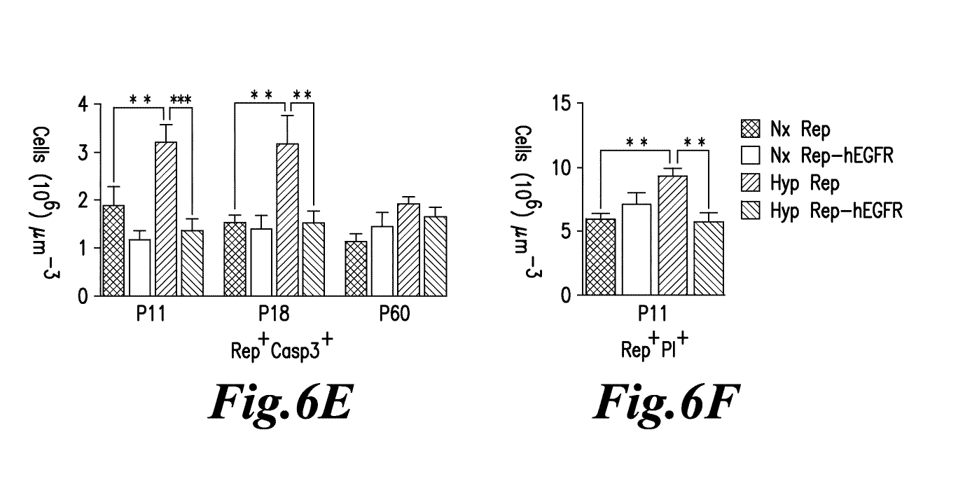

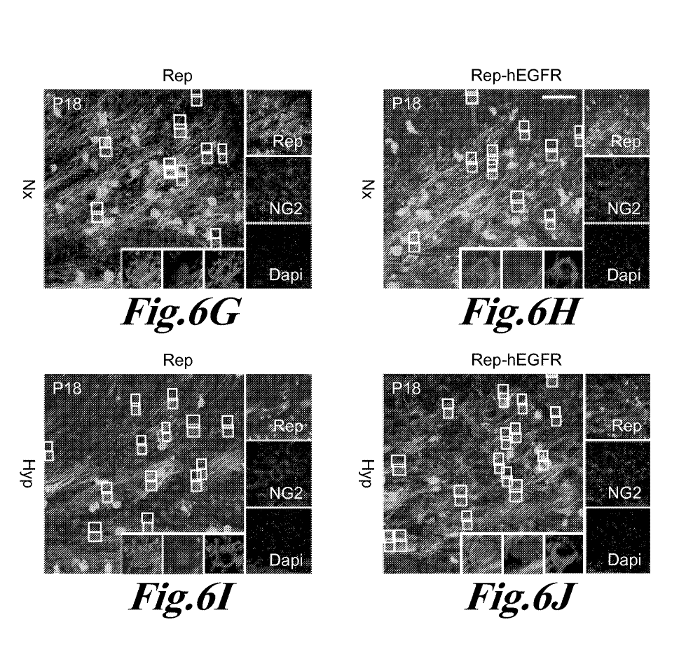

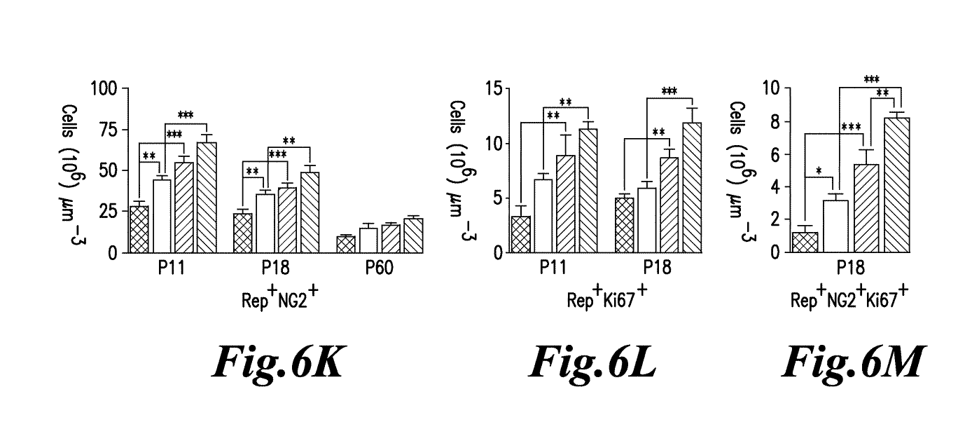

HB-EGF treatment also caused an increase in white matter Rep⁺NG2⁺ OPCs in both normoxia and hypoxia groups, and enhanced OPC proliferation²⁴ (FIG. 3e, f). To determine whether oligodendrocyte recovery was a result of increased oligodendrogenesis from this expanded pool of proliferative OPCs, we performed BrdU-pulse chase labelling of newly generated oligodendrocytes. HB-EGF increased the number of Rep⁺CCl⁺BrdU⁺ cells under normoxic and hypoxic conditions at P18 (FIG. 3g). At P30, the additive effect of hypoxia and HB-EGF was not as evident (FIG. 3g). Genetic lineage tracing of oligodendrocytes in vivo by using the platelet-derived growth factor-α receptor (PDGFαR-CreER:Z/EG) (GFP⁺) reporter mouse²⁵ (Extended Data FIG. 6 and FIG. 3h) confirmed that hypoxia and HB-EGF treatment have an additive effect on the generation of Olig2-expressing cells (Extended data FIG. 6j). Conversely, PDGFαR-driven EGFR deletion in OPCs prevented the cellular effects of HB-EGF on NG2⁺ OPCs (FIG. 3i). HB-EGF treatment after hypoxia also resulted in recovery of MBP and proteolipid protein (PLP) expression (FIG. 3j). These results indicate that, after hypoxia, HB-EGF promotes expansion of the OPC pool, and oligodendrocyte regeneration and maturation.

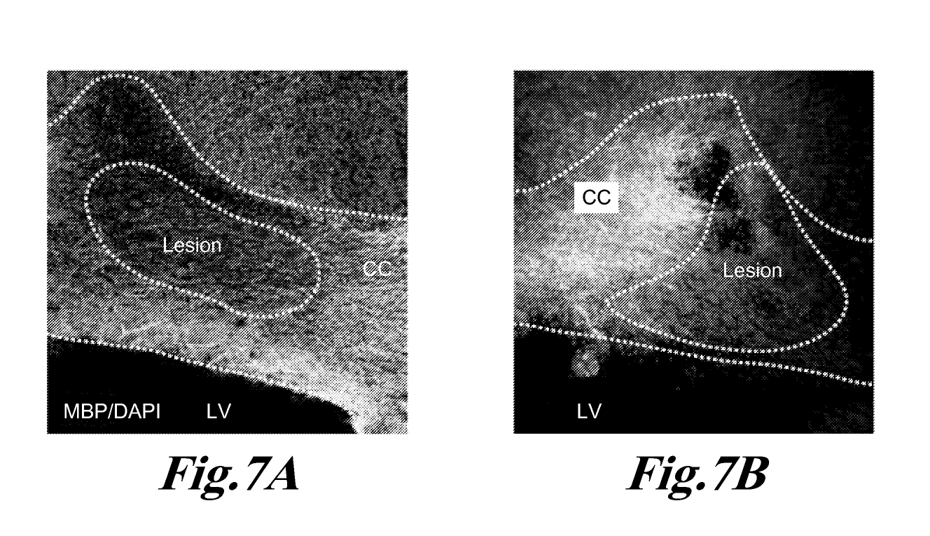

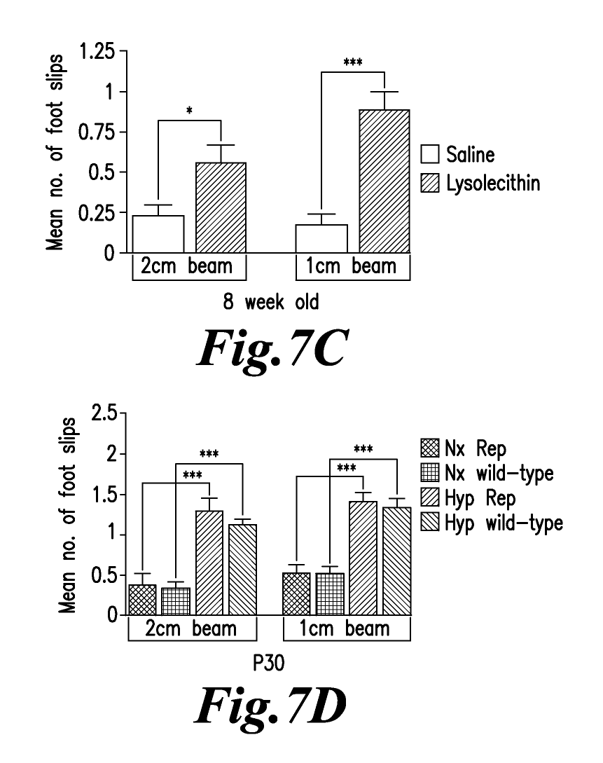

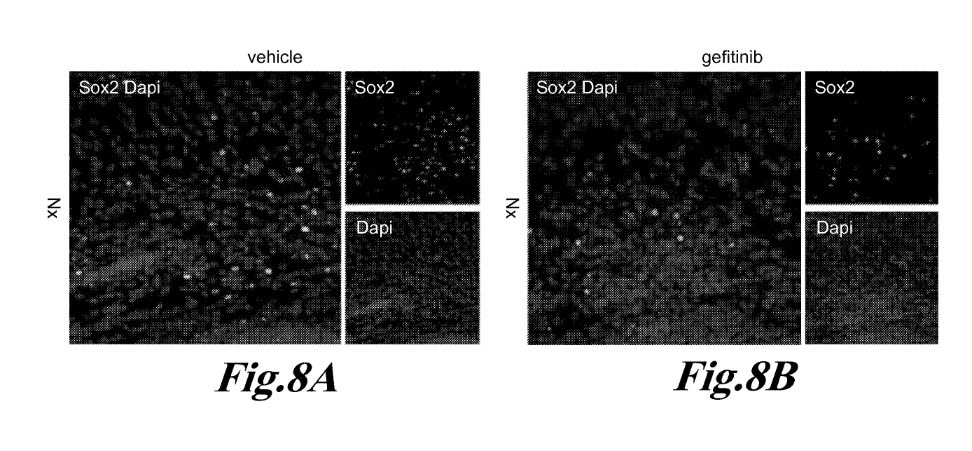

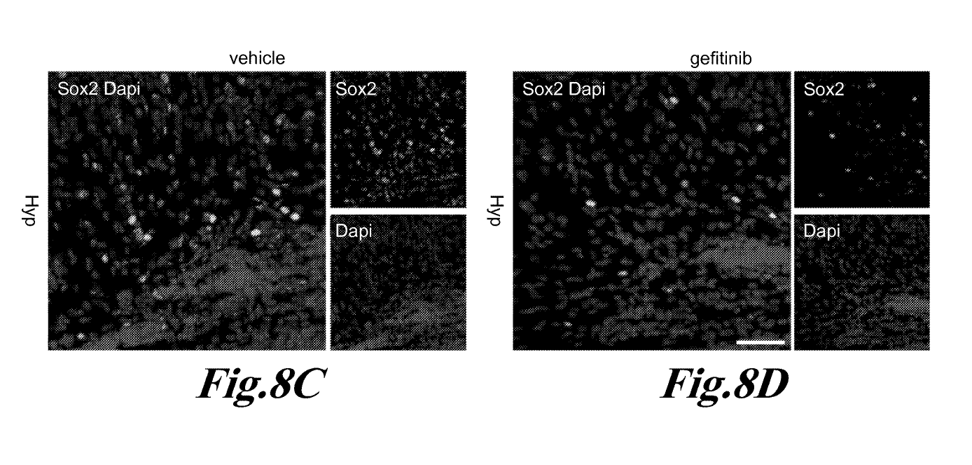

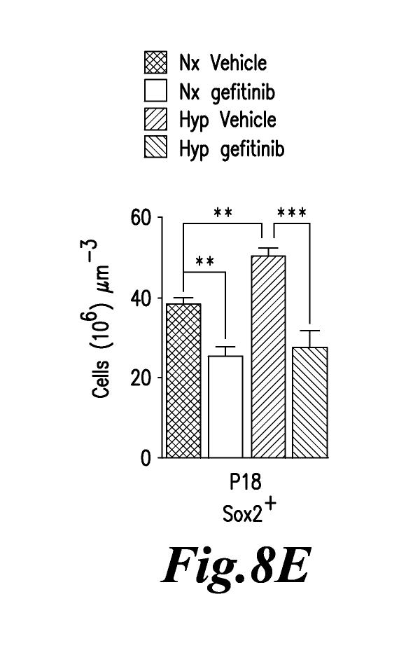

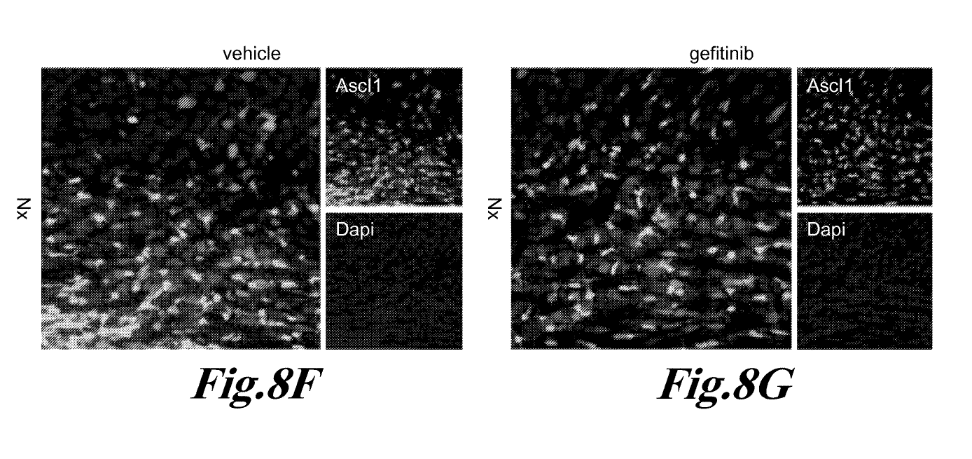

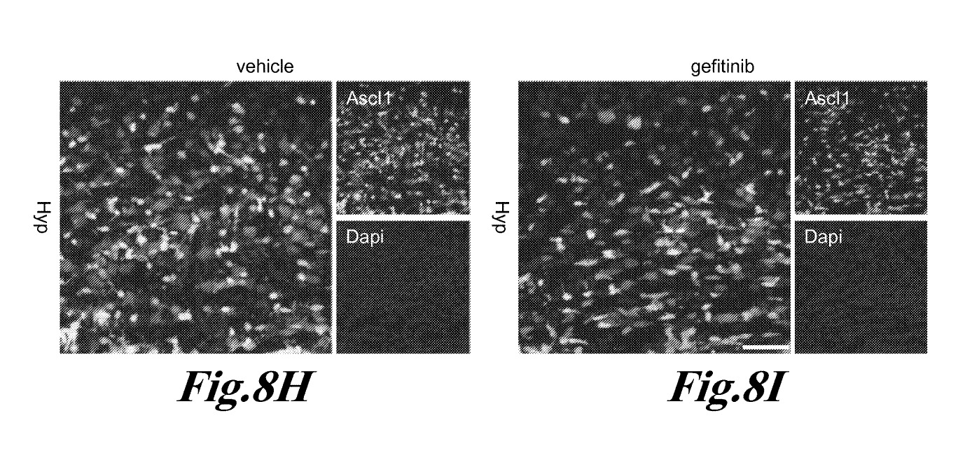

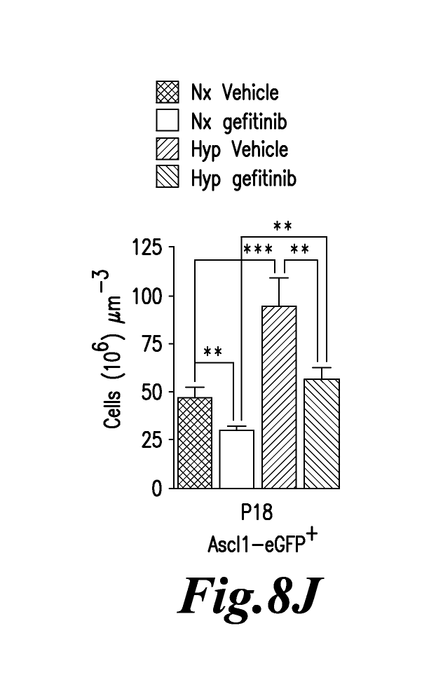

Electron microscopy analysis revealed that HB-EGF treatment rescued the increase in g ratio observed in hypoxia-treated mice and partially prevented the decrease in percentage of myelinated axons (FIG. 4a-f and Extended Data FIG. 7). Diffusion tensor imaging (DTI) demonstrated that, at P60, fractional anisotropy (FA) values are significantly decreased in the corpus callosum, cingulum and external capsule regions of hypoxia-treated mice, but not in the HB-EGF-treated hypoxia group (FIG. 4g-j). Electrophysiological analysis of extracellular compound action potentials (CAPs) demonstrated that HB-EGF prevented the decrease in amplitude of myelinated axons observed after hypoxia (FIG. 4k). Finally, analysis of N-acetylaspartate (NAA) in the white matter showed decreased levels at P18 and P30, which was prevented by HB-EGF (FIG. 4l and Extended Data FIG. 8).

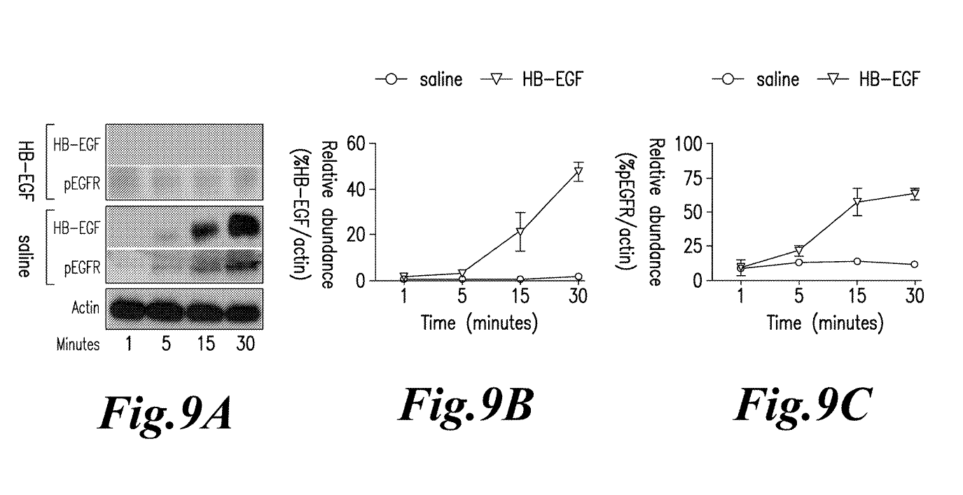

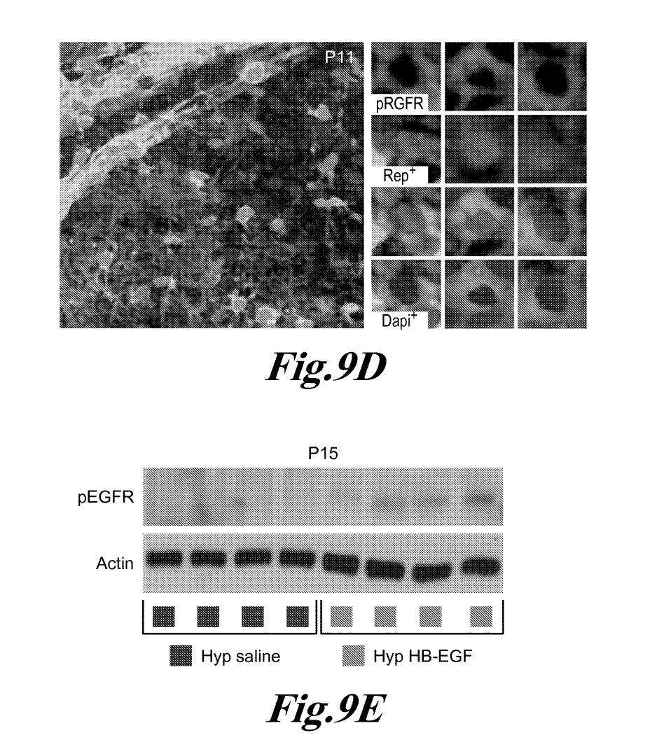

On the complex wheel, hypoxia-exposed HB-EGF-treated mice showed a similar performance to the normoxia saline-treated group (FIG. 4m). Furthermore, in the inclined beam-walking task, HB-EGF treatment completely prevented hypoxia-induced behavioural deficit, as tested on the 2-cm-width beam, and reduced the effects of hypoxia on performance on the 1-cm-width beam (FIG. 4n, o). Importantly, when HB-EGF treatment was performed at P18-P21, it had no effect on the hypoxia-induced behavioural phenotype (FIG. 4p and Extended Data FIG. 9).

Importantly, HB-EGF strongly inhibited hypoxia-induced upregulation of Notch signalling elements (Extended Data FIG. 10a-c) and functional activation of Notch in white matter Olig2⁺ oligodendrocyte lineage cells (Extended Data FIG. 10d-h), which could be at least in part responsible for delayed oligodendrocyte maturation after perinatal injury²⁶. These results are consistent with the notion that HB-EGF accelerated OPC maturation after hypoxia through inhibition of Notch¹¹, and indicates that HB-EGF treatment promotes functional recovery during a critical developmental time window for effective therapeutic intervention.

The inventors' results reveal that activating EGF/EGFR signalling promotes cellular and functional recovery after neonatal brain injury. Enhancing EGFR signalling through overexpression of the EGFR prevents DWMI, promotes the generation of new oligodendrocytes and prevents behavioural deficits in different white-matter-related tasks. Furthermore, a brief pharmacological treatment that targets endogenous EGFRs using a clinically feasible (intranasal) mode of entry during a critical window promotes cellular, developmental and myelin structural improvement, and behavioural recovery. Intranasal treatment is a plausible route to introduce sufficient HB-EGF into the brain and white matter of critically ill VPT infants.

### Examples

Data presented are from male mice on a C57BL/6 background. Mice underwent chronic perinatal hypoxia from P3-P11, as previously described^{27,28}. In the first set of experiments, Rep mice that did not express hEGFR were used as littermate controls. The EGFR antagonist gefitinib (Astra Zeneca) was administered intraperitoneally at 75 mg kg⁻¹ body weight once daily for 7 days. Normoxia and hypoxia control groups received vehicle on the same days and times. HB-EGF was administered intranasally at a concentration of 100 ng g⁻¹ body weight in 5 μl increments separated by 10 min. Equal volumes of saline were used as a vehicle control.

Animals.

The CNP-EGFP (Rep) and CNP-EGFP-hEGFR (Rep-hEGFR) strains were generated as described previously and backcrossed to a C57BL/6 genetic background greater than nine generations^{4,11,12}. The Rep-hEGFR mice used for these experiments were crossed with heterozygote Rep mice to ensure that all pups were positive for GFP, but not all expressed hEGFR. This ensured littermate controls. For experiments that required only the Rep line of mice, a Rep adult mouse was crossed with a C57BL/6 adult mouse (Jackson Laboratories). Only mice that expressed eGFP (Rep) during screening on P2 with ultraviolet goggles were used. The PDGF□R-CreERT2 line (courtesy of D. Bergles; referred to in the text as PDGFαR-CreER) was crossed with Z/EG reporter mice (Jackson Laboratories; stock number 003920) and genotyped as previously reported²⁵. Z/EG reporter mice were crossed with EGFR^{fl/fl} (ref 29) followed by PDGFαR-CreER transgenic mice. These mice were backcrossed to ensure homozygote EGFR^{fl/fl} mice, so that all mice were PDGFαR-CreER; EGFR^{fl/fl}; Z/EG. To induce Cre recombination, tamoxifen was administered at a dosage of 75 μg g⁻¹ body weight. The Ascl1^{GFP} mice (Jackson Laboratories, stock 012881; also known as Mash1) were used to determine the effects of gefitinib on white matter Ascl1-expressing cells. Unless described later or in the figure legends, only male mice were used, owing to male preterm children showing more clinically relevant injury and neurological deficits compared with females^{30,31}. All animal procedures were performed according to the Institutional Animal Care and Use Committee of the Children's National Medical Center and the Guide for the Care and Use of Laboratory Animals (National Institutes of Health).

Hypoxic Rearing.

Mice were randomly chosen to either undergo hypoxic rearing or serve as normoxia controls. The hypoxic mice were placed in a sealed chamber maintaining O₂ concentration at 10.5% by displacement with N₂ as described previously^{27,28,32}. Hypoxia began at P3 for 8 days until P11. This time window in rodent white matter oligodendrocyte development reproduces changes that occur at 23-40 weeks of gestation in the human brain⁹. A separate group of mice from the same breeding cage were used for age- and strain-matched normoxia controls. Genotyping (CNP-hEGFR; PDGFαR-CreER; Z/EG; Ascl1^{GFP}; EGFR^{fl/fl}; and Notch reporter mice) was performed at P11 by PCR of tissue obtained from the tail as previously reported^{4,11,25,29}. Time points chosen for immunohistochemistry or protein quantification were P11, P14.5, P15, P18, P30 and P60.

BrdU Administration.

The BrdU labelling protocol was performed in all mice as follows. Mice were injected intraperitoneally (i.p.) at the same time of the day with BrdU (50 μg g⁻¹ body weight) daily for 4 days (P11-P14) in the morning. In studies using HB-EGF or gefitinib, BrdU was administered 1 h before vehicle or drug administration.

Gefitinib Administration.

Gefitinib (Iressa; Tocris (Astra Zeneca)) was prepared with strong sonication in 25% dimethylsulphoxide (DMSO) and 75% sunflower seed oil at a concentration of 10 mg ml⁻¹. Male mice in each litter were randomly chosen to receive either drug or vehicle. The drug dose for this study was 75 mg kg⁻¹ day⁻¹ and was administered once daily. An equal amount of vehicle was administered to control animals. A total of seven doses of vehicle or drug were administered beginning at P12.

HB-EGF Administration.

The intranasal route allows for small molecules to rapidly enter the cerebrospinal fluid from the nasal cavity, followed by subsequent distribution to the brain and spinal cord³³⁻³⁶. Recombinant human heparin-binding epidermal growth factor constituent free (HB-EGF; R&D Biosciences) was prepared using 0.45% normal sterile saline solution at a concentration of 20 μg ml⁻¹ and stored at −20° C. Mice were randomized to the vehicle (saline) or HB-EGF group. Saline or HB-EGF was administered intranasally at no more than 5 μl increments 5-10 min apart for a total of 100 ng g⁻¹. The mouse was held ventral-side up, and a small-modified 27-French catheter was inserted into either nare. Saline or drug was slowly administered and the mouse was held for 1-2 min to ensure absorption. Drug or saline was administered every 12 h beginning on the evening of P11.

Immunohistochemistry and Antibodies.

Freshly cut, free-floating brain sections (40 □m thick) from P11-P60 mice were prepared as described previously^{3,11,27,32}. Primary antibody dilutions were 1:500 for anti-BrdU (Accurate), anti-NG2 (Millipore), anti-Olig2 (Millipore), anti-Ki67 (Vector), anti-APC (also referred as CC1; Millipore) and anti-cleaved caspase-3 (caspase-3; Millipore); 1:250 for anti-MBP (Covance); and 1:500 anti-EGFR phosphorylated Tyr 1068 (Novus Biologicals). Sections were incubated at room temperature (22-25° C.) for 1-3 h, followed by overnight at 4° C. in primary antibodies diluted in 0.1 M PBS containing 0.1% Triton X-100 and 5% normal goat serum (vol/vol). Three washes were performed with cold 1 PBS before secondary antibodies being administered. The secondary antibodies (1:200) used were AlexaFluor 488, AlexaFluor 546 and AlexaFluor 633 conjugated goat anti-rabbit, anti-rat or anti-mouse IgG (Invitrogen). Sections were incubated with secondary antibodies for 1 h at room temperature, followed by three 1×PBS washes. Sections were treated with 4′,6-diamidino-2-phenylindole (DAPI) (Sigma-Aldrich) for 10-15 min at room temperature and mounted with Mowiol. Human tissue was obtained from a deceased 3-day-old infant born at 36-week gestation with approval from the Children's National Institutional Review Board. Sections of corpus callosum and periventricular region were removed after fixation of the brain for 2 weeks in formalin solution. Tissue was kept in PBS for 1 week with daily changes of PBS solution to remove excess formalin. The brain was then placed in 20% glycerol solution for 24 h. Freshly cut, free-floating sections (40 μm thick) were made on a sliding microtome. Sections were immunostained as described earlier.

Microscopy and Cell Counting.

All fluorescent images were taken on a Zeiss LSM confocal laser-scanning microscope with sequential scanning mode using ×40 oil objectives. Microscopy and cell counting were performed as recently described^{37,38}. Z-stack images of 1-μm-thick single planes were captured throughout the entire thickness of the slice and each cell was analysed using a Zeiss LSM Image Browser (version 4.2) in its entire z-axis to exclude false double labelling due to overlay of signals from different cells. Four different laser lines were used to perform image localization of fluorescein isothiocyanate (FITC) (488 nm excitation; 522/35 emission filter), CY3 (560 nm excitation; 605/32 emission filter), Cy5 (647 nm excitation; 680/32 emission filter) and DAPI (400 nm excitation). Data acquisition and processing were controlled by modified LSM software. Analysis of immunofluorescence was performed on a confocal z-stack as previously described^{37,38}. Cells were counted in 225×225×10 μm (X, Y, Z) images for cells per volume quantifications. Data were obtained from an average of six tissue sections per mouse per immunostain. Analysis of subcortical white matter—rostral to the hippocampus—was performed in the corpus callosum, cingulum and external capsule.

Cells were counted in a blinded fashion. The merged image for each confocal z-stack was analysed and positive immunofluorescence identified for each individual channel. Merged images were processed in Photoshop CS5.5 with minimal manipulation of contrast.

Propidium iodide. To assess loss of membrane integrity and increased plasmalemma permeability in vivo, propidium iodide was used as demonstrated previously³⁹. Propidium iodide (10 mg ml⁻¹; Sigma) was diluted in 0.9% NaCl and 1 mg kg⁻¹ was administered i.p. 1 h before mice were killed. As described earlier, mice were perfused, brains were collected from all groups and free-floating brain sections (40 □m thick) were prepared. Sections were washed, incubated with DAPI for 10 min, washed and then mounted on a slide. Propidium iodide (PI) emits bright red fluorescence when bound to RNA or DNA. Confocal microscopy was used to visualize the GFP⁺PI⁺DAPI⁺ cells.

Western Blot Analysis.

For western blot analysis of white matter lysates, the subcortical white matter was dissected on ice-cold medium from 300-400-μm-thick sections as previously described^{4,27,32.} Briefly, brains were sliced coronally and only sections anterior to the hippocampus were used. Using Roboz—a fine-straight and fine-angled microdissecting forceps under a dissecting microscope—the cortex was dissected away leaving the subcortical white matter attached to the striatum. The white matter was then easily pushed away from the striatum, leaving only white matter tissue. The dissected white matter was rinsed with ice-cold PBS, and then lysed on ice in 150-200 μl of RIPA lysis buffer with protease inhibitors. Protein concentrations were determined by using the Bradford protein assay kit (Bio-Rad). Western blot analysis was performed using 10-40 μg of total cell lysates. Proteins were resolved on 4-20% Tris glycine gels (NuSep) and transferred to Immobilon PVDF membranes in transfer buffer overnight at 4° C. Membranes were blocked for 1 h in 5% milk in Tris-buffered saline-Tween 20 (TBST), then incubated at 4° C. overnight with primary antibodies diluted in 5% milk in TBST: 1:5,000 for anti-MBP (Covance), anti-CNP (Covance) and anti-actin (Millipore); 1:1,000 for anti-PLP (Abeam); 1:1,000 for anti-HB-EGF (Santa Cruz); 1:1,000 for anti-Delta1 (Santa Cruz); 1:1,000 for anti-NICD (Iowa Hybridoma Bank C17.9C6); and 1:4,000 for anti-aspartoacylase (ThermoScientific)). The membranes were then washed in TBST three times for 10-15 min at room temperature followed by the addition of either horseradish-peroxidase-conjugated goat polyclonal anti-rabbit IgG (Santa Cruz) for polyclonal primary antibodies, or horseradish-peroxidase-conjugated goat anti-mouse (Santa Cruz) for mouse monoclonal primary antibodies diluted in 5% milk in TBST. For phosphorylated EGFR (Novus Biologicals), 5% bovine serum albumin (BSA) in TBST was used as a block and for primary antibody incubation overnight. For all western blots, chemiluminescent signals were detected using Pierce ECL western blotting substrate. X-ray films were scanned using an Agfa T1200 scanner and densitometric measurements were obtained using ImageJ software http://_rsb.info.nih.gov/ij/. Western blots were obtained from the white matter of 3-6 male mice in each group. Densitometric measurements were obtained using ImageJ software averaged as previously described^{4,27,32}.

EGF ELISA.

WM was grossly dissected on ice-cold 1×PBS from P11, P15 and P18 Rep and Rep-hEGFR normoxia and hypoxia mice as described earlier. Assay procedure was performed according to the manufacturer's instructions (R&D Systems, Mouse EGF Quantikine ELISA Kit). Experiments were performed in triplicates and averaged.

FACS.

White matter microdissected tissue from hypoxia-exposed P15 Rep⁺ (CNP-EGFP⁺) vehicle- or HB-EGF-treated mice were FACS purified. Two-to-three male and female brains were pooled for each sample (individual n). Tissue was dissociated into single-cell suspensions as previously described^{11,40}, and analysed for light forward and side scatter using a FACSAria instrument (BD Bioscience). The collected Rep⁺ cells were used for western blot analysis.

Electron Microscopy.

Mice at P30 and P60 were perfused with 4% paraformaldehyde containing 10% picric acid and 5% glutaraldehyde and post-fixed for 2 weeks⁴¹. Brains were sectioned and prepared in groups at the same time as previously described^{32,41}. Sagittal sections of white matter were examined with a JEOL transmission electron microscope (JEM-1400), and pictures were taken with a Gatan SC 1000 ORIUS CCD camera. Measurements and image processing was performed using ImageJ. Myelin thickness was calculated from the average of radial measurements at four points per sheath, avoiding areas of tongue processes or fixation artefact^{41,42}. Axon diameters were calculated from measurement of the axon circumference. Axons with diameters typical of unmyelinated fibres (<0.3 μm) were excluded from analysts^{41,42}. The extent of myelination was quantitatively compared by determining g ratios, which were calculated by dividing the diameter of the axon by the diameter of the entire myelinated fibre, as previously described⁴¹⁻⁴³. Blind measurements of the groups were made. At least 100 axons were measured for each brain.

LPC-Induced Demyelination.

Bilateral demyelination was performed in adult male and female C57BL/6 (8 weeks old) mice after deep ketamine/xylazine anaesthesia (10 mg g⁻¹ body weight). Mice were placed in a modified stereotaxic frame (Stoetling) and 2 μl of 2% lysolecithin (EMD Chemicals, LPC) solution (vol/vol) and/or 0.9% NaCl (vol/vol) was injected bilaterally into the corpus callosum using a Hamilton micropette (Stoetling). Injection time lasted for 5 min to reduce reflux along the needle track. The needle was then slowly withdrawn over a 5 min period. Stereotaxic coordinates for the corpus callosum were taken from Bregma (0.26 mm caudal, 1.0 mm lateral and 3.0 mm ventral). Inclined-beam walking behavioural test testing began 5 days after surgery in both groups of mice as described later. Bilateral demyelination was confirmed after testing by perfusing the mice as described earlier and immunohistochemical analysis of the corpus callosum was performed using anti-MBP. Only mice that displayed clear bilateral lesions on microscopic examination were included in the behavioural analysis. Three (n=3) mice were excluded from the study because bilateral white matter demyelination was not clearly evident.

DTI Analysis.

Mice (P60) used for DTI were perfused and imaged as previously described⁴⁴. One hour before DTI scans the brains were soaked 3 times for 10 min each time in 10 ml PBS to remove the PFA solution. The brains were placed into a custom-built MRI-compatible tube filled with Fluorinert—an MRI susceptibility-matching fluid (Sigma-Aldrich). The DTI data sets were obtained on a 9.4 T horizontal bore magnet (Bruker) with a custom-made ¹H radio frequency coil. The DTI experiments were performed using the Stejskal-Tanner spin-echo diffusion-weighted sequence with a diffusion gradient of 5 ms and a delay between the two diffusion gradients of 15 ms. Twenty-four contiguous coronal slices of 0.5 mm thickness were acquired using a repetition time (TR) of 2 s and an echo time (TE) of 25.1 ms. Two Shinnar-Le Roux (SLR) pulses of 1 ms each were used for excitation and inversion, respectively. Twenty averages were acquired for each slice and the 128×64 pixel resolution images were zero-filled to 256×256 pixel resolution, resulting in an in-plane spatial resolution of 100 μm×100 mm². Sixteen different images were acquired for each slice, 15 corresponding to various non-collinear diffusion-weighting directions with b=1,000 s mm⁻² and one with no diffusion weighting. DTI processing and analysis was performed blindly as described previously^{44,45}.

CAPs.

Compound action potential (CAP) recordings were performed in all four groups at P30 and P60 using methods previously described^{4,38}. Briefly, after mice were killed, coronal slices 400 μm in thickness were obtained using a VT1000S vibratome (Leica) in ice-cold slicing solution (85 mM NaCl, 2.5 mM KCl, 25 mM NaHCO₃, 1.25 mM NaH₂PO₄, 0.5 mM CaCl₂, 7 mM MgCl₂, 25 mM glucose, 75 mM sucrose). Slices were placed in recording solution (125 mM NaCl, 2.5 mM KCl, 26 mM NaHCO₃, 1.25 mM NaH₂PO₄, 2.5 mM CaCl₂, 1.3 mM MgCl₂, 11 mM glucose, pH 7.4) bubbled in 95% O₂ and 5% CO₂, and maintained at 37° C. for 1 h then kept in the same solution at room temperature until recording. CAP recordings were performed on five slices corresponding to sections 13-18 (Bregma 1.32 to −0.94 mm) of the National Institutes of Mental Health's mouse brain atlas (http://_www.mbl,org/atlas170/atlas170_frame.html). Slices were placed in a recording chamber superfused with oxygenated recording solution at a flow rate of 2 ml min⁻¹ and viewed using the ×10 objective of an Olympus BX61WI microscope. CAP recordings were obtained using an FHC concentric bipolar stimulating electrode and an extracellular field electrode with a tip resistance of 1 M. The stimulating and recording electrodes were placed ˜2 mm apart (˜1 mm on each side of the midline) in the corpus callosum of subcortical white matter, and a constant stimulus was delivered for each recording in current clamp. A single pulse protocol was used with a pulse frequency of 200 Hz and a period of 5 ms. Fifty sweeps were recorded, averaged and used in the analysis for CAP amplitude. Two distinguishable downward waves were evident for each CAP recording, with the first corresponding to rapidly propagating myelinated (M) axons and the second to slower propagating unmyelinated (UM) axons^{4,38,46}.

¹H-NMR.

Brains from all groups were collected at P11, P18 and P30. The brains were removed and placed on dry ice. The white matter was dissected, snap frozen in liquid nitrogen (total time <60 s), and stored in a −80° C. freezer until extraction. Samples for ¹H-NMR spectroscopy were prepared as previously published⁴⁷. Each frozen sample was homogenized in 1 ml of 7% perchloric acid and centrifuged for 10 min at 4° C. and 4,600 g-force. Supernatants were transferred to separate tubes and pellets were re-extracted. Combined supernatants were neutralized with KOH, centrifuged and lyophilized. Lyophilized samples were dissolved in 0.8 ml of 99% D₂O and pH was adjusted to 7.0. Fully relaxed ¹H-NMR spectra were acquired on Varian 500 with the following parameters: 90° pulse angle, an acquisition time of 1.36 s, relaxation delay of 17 s, total number of 800 scans per sample. Low-power pre-saturation pulse at water frequency was used to achieve water suppression. Obtained spectra were analysed using MestReNova software (version 8.1; Mestrelab Research) and the amounts of metabolites were quantified from integrals of the peak areas corrected for number of protons and using 2,2,3,3-D(4)-sodium-3-trimethylsilylpropionate as an internal control.

Behavioral Testing.

Each behavioural experiment was performed in separate naive mice that had not undergone any previous behavioral testing. The complex running wheel task was performed as previously described¹⁴⁻¹⁶. At P45, naive mice that had not undergone any previous behavioral testing were individually housed in a modified cage equipped with a running wheel attached to an optical sensor to constantly detect the number of wheel revolutions per time interval (minute). Animals were kept on a regular 12 h light/dark cycle. Food and water were made available ad libitum. During the first 2 weeks, a training wheel with all 38 rungs was present, allowing for normalization of running behavior. On the third week (day 15; age P60), the regular training wheel was replaced with a complex wheel of the same diameter with 22 rungs missing in an alternative pattern. Using Activity Wheel Monitoring Software (Lafayette Instruments), wheel revolutions were recorded each day and exported to a Microsoft Excel file in which daily total distance traveled and maximum daily velocity were calculated. All mice showed spontaneous running behaviour and no mice were excluded from this study. The inclined beam-walking task was performed as previously described^{17,18}. Two elevated 80 cm in length wooden beams were placed at a 30° angle. One beam was 2 cm in width and the other was 1 cm in width. A dark box with bedding was at the end of the incline and served as a target for the mouse to reach. A blinded experimenter observing and recording from above assessed mouse performance by documenting the number of foot slips (either hind legs or front legs) and the time to traverse the beam¹⁸. In pilot studies, we determined that hypoxia-exposed Rep mice less than 30 days of age—or on a beam that was inclined more than 30°—were unable to perform this task (data not shown). To confirm whether this sensorimotor task is dependent on subcortical white matter, bilateral LPC- or 0.9% saline-injected adult mice were tested on day 5 after surgery (described earlier). Bilateral demyelination was confirmed after testing by removal of brains and immunohistochemical analysis of corpus callosum. Only mice that had clear bilateral lesions on microscopic examination were included in the behavioural analysis (n=3 mice were excluded due to failure in demyelination).

Statistics.

All data in histograms are presented as averages ±s.e.m. All cell counting and western blot data were statistically compared using one-way ANOVA to determine whether overall differences exist across study groups. Comparisons between specific groups were treated as unplanned comparisons, which were adjusted using a Bonferroni correction. A two-tailed type 1 error (P value <0.05) was used to determine statistical significance. Each experiment evaluated outcomes in four groups. The Bonferroni correction was applied for the following comparisons: normoxia (Nx)ₐ versus Nx_{b}; Nxₐ versus hypoxia (Hyp)ₐ; and Nx_{b} versus Hyp_{b}. The two hypoxia groups were compared post-hoc if the one-way ANOVA was significant using a two-tailed unpaired t-test (Hypₐ versus Hyp_{b}) with two-tailed type 1 error set at P=0.05. For the electron microscopy data, a one-way ANOVA was used to compare the g ratio for each mouse in each of the four respective groups. If significance was found, then a two-tailed unpaired t-test with two-tailed type 1 error set at P=0.05 was used to make the following comparisons: Nxₐ versus Hypₐ; Hypₐ versus Hyp_{b}; Nxₐ versus Nx_{b}; and Nx_{b} versus Hyp_{b}. Scatter plots of g ratios of individual fibres in relation to axon diameter are shown comparing the groups outlined earlier. The average FA anisotropy for each mouse in each group was compared using a one-way ANOVA as described earlier. The Bonferroni correction was applied for the following comparisons: Nx saline versus Hyp saline; Nx saline versus Hyp HB-EGF; and Hyp saline versus Hyp HB-EGF. For the complex running wheel data, we used a longitudinal linear regression analysis to compare slopes (trajectories) of change. Post-hoc testing was also performed between the four groups for each individual day. For the beam-walking behavioral results, the number of foot slips, the time to traverse the beam and the size of the beam were analysed using a Poisson multiple regression analysis, which allowed us to overcome the lack of normality in count-type data and account for other variables to compare the rate of foot slips. The one-way ANOVA with post hoc comparisons was performed using GraphPad Prism 5.0 (for Mac). All histograms and scatter plots in this manuscript were created with GraphPad Prism.

### REFERENCES

1. Ment, L. R., Hirtz, D. & Huppi, P. S. Imaging biomarkers of outcome in the developing preterm brain. Lancet Neurol. 8, 1042-1055 (2009).
2. Woodward, L. J., Anderson, P. J., Austin, N. C., Howard, K. & Inder, T. E. Neonatal MRI to predict neurodevelopmental outcomes in preterm infants. N. Engl. J Med. 355, 685-694 (2006).
3. Buser, J. R. et al. Arrested preoligodendrocyte maturation contributes to myelination failure in premature infants. Ann. Neurol. 71, 93-109 (2012).
4. Aguirre, A., Dupree, J. L., Mangin, J. M. & Gallo, V. A functional role for EGFR signaling in myelination and remyelination. Nature Neurosci. 10, 990-1002 (2007).
5. Anjari, M. et al. The association of lung disease with cerebral white matter abnormalities in preterm infants. Pediatrics 124, 268-276 (2009).
6. Skranes, J. et al. Clinical findings and white matter abnormalities seen on diffusion tensor imaging in adolescents with very low birth weight. Brain 130, 654-666 (2007).
7. Scafidi, J., Fagel, D. M., Ment, L. R. & Vaccarino, F. M. Modeling premature brain injury and recovery. Int. J. Dev. Neurosci. 27, 863-871 (2009).
8. Silbereis, J. C., Huang, E. J., Back, S. A. & Rowitch, D. H. Towards improved animal models of neonatal white matter injury associated with cerebral palsy. Dis. Model. Mech. 3, 678-688 (2010).
9. Back, S. A. et al. Late oligodendrocyte progenitors coincide with the developmental window of vulnerability for human perinatal white matter injury. J Neurosci. 21, 1302-1312 (2001).
10. Ivkovic, S., Canoll, P. & Goldman, J. E. Constitutive EGFR signaling in oligodendrocyte progenitors leads to diffuse hyperplasia in postnatal white matter. J. Neurosci. 28, 914-922 (2008).
11. Aguirre, A., Rubio, M. E. & Gallo, V. Notch and EGFR pathway interaction regulates neural stem cell number and self-renewal. Nature 467, 323-327 (2010). </jrn>
12. Yuan, X. et al. Expression of the green fluorescent protein in the oligodendrocyte lineage: a transgenic mouse for developmental and physiological studies. J. Neurosci. Res. 70, 529-545 (2002).
13. Aguirre, A., Rizvi, T. A., Ratner, N. & Gallo, V. Overexpression of the epidermal growth factor receptor confers migratory properties to nonmigratory postnatal neural progenitors. J. Neurosci. 25, 11092-11106 (2005).
14. Hibbits, N., Pannu, R., John Wu, T. & Armstrong, R. C. Cuprizone demyelination of the corpus callosum in mice correlates with altered social interaction and impaired bilateral sensorimotor coordination. ASN Neuro 1, e00013 (2009).
15. Liebetanz, D. & Merkler, D. Effects of commissural de- and remyelination on motor skill behaviour in the cuprizone mouse model of multiple sclerosis. Exp. Neurol. 202, 217-224 (2006).
16. Schalomon, P. M. & Wahlsten, D. Wheel running behavior is impaired by both surgical section and genetic absence of the mouse corpus callosum. Brain Res. Bull. 57, 27-33 (2002).
17. Brooks, S. P. & Dunnett, S. B. Tests to assess motor phenotype in mice: a user's guide. Nature Rev. Neurosci. 10, 519-529 (2009).
18. Carter, R. J., Morton, J. & Dunnett, S. B. in Current Protocols in Neuroscience (John Wiley & Sons, 2001).
19. Heimberger, A. B. et al. Brain tumors in mice are susceptible to blockade of epidermal growth factor receptor (EGFR) with the oral, specific, EGFR-tyrosine kinase inhibitor ZD1839 (Iressa). Clin. Cancer Res. 8, 3496-3502 (2002).
20. Jin, K. et al. Cerebral neurogenesis is induced by intranasal administration of growth factors. Ann. Neurol. 53, 405-409 (2003).
21. Illum, L. Transport of drugs from the nasal cavity to the central nervous system. Eur. J. Pharm. Sci. 11, 1-18 (2000).
22. Dhuria, S. V., Hanson, L. R. & Frey, W. H. Intranasal delivery to the central nervous system: mechanisms and experimental considerations. J. Pharm. Sci. 99, 1654-1673 (2010).
23. Cantarella, C., Cayre, M., Magalon, K. & Pascale, D. Intranasal HB-EGF administration favors adult SVZ cell mobilization to demyelinated lesions in mouse corpus callosum. Dev. Neurobiol. 68, 223-236 (2008).
24. Sugiarto, S. et al. Asymmetry-defective oligodendrocyte progenitors are glioma precursors. Cancer Cell 20, 328-340 (2011).
25. Kang, S. H., Fukaya, M., Yang, J. K., Rothstein, J. D. & Bergles, D. E. NG2⁺ CNS glial progenitors remain committed to the oligodendrocyte lineage in postnatal life and following neurodegeneration. Neuron 68, 668-681 (2010).
26. Zhang, Y. et al. Notchl signaling plays a role in regulating precursor differentiation during CNS remyelination. Proc. Natl Acad. Sci. USA 106, 19162-19167 (2009).
27. Raymond, M., Li, P., Mangin, J.-M., Huntsman, M. & Gallo, V. Chronic perinatal hypoxia reduces glutamate-aspartate transporter function in astrocytes through the Janus kinase/signal transducer and activator of transcription pathway. J. Neurosci. 31, 17864-17871 (2011).
28. Bi, B. et al. Cortical glial fibrillary acidic protein-positive cells generate neurons after perinatal hypoxic injury. J. Neurosci. 31, 9205-9221 (2011).
29. Lee, T.-C. & Threadgill, D. W. Generation and validation of mice carrying a conditional allele of the epidermal growth factor receptor. Genesis 47, 85-92 (2009).
30. Ment L. R. et al. Change in cognitive function over time in very low-birth-weight infants. J. Am. Med. Assoc. 289, 705-711 (2003).
31. Ment, L. R. et al. Longitudinal brain volume changes in preterm and term control subjects during late childhood and adolescence. Pediatrics 123, 503-511 (2009).
32. Jablonska, B. et al. Oligodendrocyte regeneration after neonatal hypoxia requires FoxO1-mediated p27Kip1 expression. J Neurosci. 32, 14775-14793 (2012).
33. Born, J. et al. Sniffing neuropeptides: a transnasal approach to the human brain. Nature Neurosci. 5, 514-516 (2002).
34. Thorne, R. G., Pronk, G. J., Padmanabhan, V. & Frey Ii, W. H. Delivery of insulin-like growth factor-I to the rat brain and spinal cord along olfactory and trigeminal pathways following intranasal administration. Neuroscience 127, 481-496 (2004).
35. Ross, T. M. et al. Intranasal administration of interferon beta bypasses the blood-brain barrier to target the central nervous system and cervical lymph nodes: a non-invasive treatment strategy for multiple sclerosis. J. Neuroimmunol. 151, 66-77 (2004).
36. Fletcher, L. et al. Intranasal delivery of erythropoietin plus insulin-like growth factor-I for acute neuroprotection in stroke. J. Neurosurg. 111, 164-170 (2009).
37. Ishibashi, N. et al. White matter protection in congenital heart surgery. Circulation 125, 859-871 (2012).
38. Ritter, J. et al. Neonatal hyperoxia exposure disrupts axon-oligodendrocyte integrity in the subcortical white matter. J. Neurosci. 33, 8990-9002 (2013).
39. Whalen, M. J. et al. Acute plasmalemma permeability and protracted clearance of injured cells after controlled cortical impact in mice. J. Cereb. Blood Flow Metab. 28, 490-505 (2008).
40. Aguirre, A. & Gallo, V. Postnatal neurogenesis and gliogenesis in the olfactory bulb from NG2-expressing progenitors of the subventricular zone. J. Neurosci. 24, 10530-10541 (2004).
41. Furusho, M., Dupree, J. L., Nave, K.-A. & Bansal, R. Fibroblast growth factor receptor signaling in oligodendrocytes regulates myelin sheath thickness. J. Neurosci. 32, 6631-6641 (2012).
42. Zhou, Y.-X., Pannu, R., Le, T. Q. & Armstrong, R. C. Fibroblast growth factor 1 (FGFR1) modulation regulates repair capacity of oligodendrocyte progenitor cells following chronic demyelination. Neurobiol. Dis. 45, 196-205 (2012).
43. Liu, J. et al. Impaired adult myelination in the prefrontal cortex of socially isolated mice. Nature Neurosci. 15, 1621-1623 (2012).
44. Duque, A. et al. Neuroanatomical changes in a mouse model of early life neglect. Brain Struct. Funct. 217, 459-472 (2012).
45. Chahboune, H. et al. Hypoxic injury during neonatal development in murine brain: correlation between in vivo DTI findings and behavioral assessment. Cereb. Cortex 19, 2891-2901 (2009).
46. Crawford, D. K., Mangiardi, M. & Tiwari-Woodruff, S. K. Assaying the functional effects of demyelination and remyelination: revisiting field potential recordings. J Neurosci. Methods 182, 25-33 (2009).
47. Scafidi, S. et al. Metabolism of acetyl-1-carnitine for energy and neurotransmitter synthesis in the immature rat brain. J. Neurochem. 114, 820-831 (2010).

1. A method for treating a subject having a brain injury caused by or associated with hypoxia comprising administering heparin-binding EGF-like growth factor, a functional fragment thereof, or a variant thereof to said subject.

2. The method of claim 1, wherein the subject is a neonate.

3. The method of claim 1, wherein the subject is a preterm infant.

4. The method of claim 1 wherein the subject is a preterm infant less than 32-weeks gestation.

5. The method of claim 1, wherein the subject has diffuse white matter injury (“DWMI”).

6. The method of claim 1, wherein the subject exhibits neurodevelopmental impairment.

7. The method of claim 1, wherein the heparin-binding EGF-like growth factor, a functional fragment thereof, or a variant thereof was produced recombinantly.

8. The method of claim 1, comprising administering a variant or functional fragment of heparin-binding EGF-like growth factor that has an amino acid sequence that is at least 90% identical to Accession No. NP_001936, version NP_001936.1 (SEQ ID NO: 2).

9. The method of claim 1, comprising administering a variant or functional fragment of heparin-binding EGF-like growth factor that has an amino acid sequence that is at least 95% identical to that described by Accession No. NP_001936, version NP_001936.1 (SEQ ID NO: 2).

10. The method of claim 1, comprising administering an EGF-like growth factor comprising the amino acid sequence of SEQ ID NO: 2.

11. The method of claim 1, wherein heparin-binding EGF-like growth factor, a functional fragment thereof, or a variant thereof is administered within 0, 0.25, 0.5, 1, 2, 3, 4, 5, 6, 7, 8, 9, 10, 12, 15, 16, 20, 24, 30, 36, 40, 48, 50, 60 or 72 hrs of brain injury.

12. The method of claim 1, comprising administering 10 to 1,000 mg/kg of heparin-binding EGF-like growth factor, a functional fragment thereof, or a variant thereof to the subject.

13. The method of claim 1, comprising administering the heparin-binding EGF-like growth factor, a functional fragment thereof, or a variant thereof into the central nervous system.

14. The method of claim 1 comprising administering the heparin-binding EGF-like growth factor, a functional fragment thereof, or a variant thereof intranasally.

15. A method for diminishing ultrastructural abnormalities caused by or associated with hypoxia in subject comprising administering heparin-binding EGF-like growth factor, a functional fragment thereof, or a variant thereof to said subject.

16. The method of claim 15, wherein the subject is a neonate.

17. The method of claim 15, wherein the subject is a preterm infant.

18. A method for decreasing oligodendroglia death and/or enhancing generation of new oligodendrocytes from progenitor cells in a subject comprising administering heparin-binding EGF-like growth factor, a functional fragment thereof, or a variant thereof to said subject.

19. The method of claim 18, wherein said subject is a neonate who has been subjected to a hypoxic state, who is hypoxic, or who is at risk of hypoxia.

20. The method of claim 18, wherein said subject is a preterm infant who has been subjected to a hypoxic state, who is hypoxic, or who is at risk of hypoxia.

21. A method for alleviating behavioral deficits associated with hypoxic brain injury on a white-matter-specific paradigm or promoting functional recovery in a subject comprising administering heparin-binding EGF-like growth factor, a functional fragment thereof, or a variant thereof to said subject.

22. The method of claim 21, wherein said subject is a neonate who has been subjected to a hypoxic state, who is hypoxic, or who is at risk of hypoxia.

23. The method of claim 21, wherein said subject is a preterm infant who has been subjected to a hypoxic state, who is hypoxic, or who is at risk of hypoxia.

24. A composition comprising heparin-binding EGF-like growth factor, a functional fragment thereof, or a variant thereof in combination with a solution, carrier or excipient that facilitates its uptake by the CNS after intranasal administration.

25. An intranasal spray or aspirator device comprising the composition of claim 24 and, optionally, pump, atomizer, a channel or conduit, and/or storage container which may be pressurized or unpressurized.
